SEQUENCE LISTING (64 sequences)

SEQ ID NO: 1 - DNA, 159 bp, synthetic construct
  source 1..159: mol_type = other DNA; note = artificial miRNA
aattgtcagcaataaacgccacccactctctttgcgcataggtacgttcgcgtccaccacccaccctgaactacgttagattcacgacacggtcggttcggtatccagaaaaccttgacctatgcgcaaagagagtgggtgtcggtatttgctgaagtg

SEQ ID NO: 2 - DNA, 95 bp, synthetic construct
  source 1..95: mol_type = other DNA; note = amiR4416-5p STTM
atacgcgtttctactctcacccagttgttgttgttatggtctaatttaaatatggtctaaagaagaagaatgatacgcgtttctactctcaccca

SEQ ID NO: 3 - DNA, 24 bp, synthetic construct
  source 1..24: mol_type = other DNA; note = miR4416-5p STTM
  misc_feature 12..14: note = CTA bulge
gatacgcgtttctactctcaccca

SEQ ID NO: 4 - DNA, 21 bp, synthetic construct
  source 1..21: mol_type = other DNA; note = miR4416-5p with a deletion
  misc_feature 11..12: note = location of deletion or insert as compared to wild-type sequence
gatacgcgtttctctcaccca

SEQ ID NO: 5 - DNA, 96 bp, synthetic construct
  source 1..96: mol_type = other DNA; note = miR4416-3p STTM
cctaggtgagactagcgacccgtagttgttgttgttatggtctaatttaaatatggtctaaagaagaagaatcctaggtgagactagcgacccgta

SEQ ID NO: 6 - DNA, 24 bp, synthetic construct
  source 1..24: mol_type = other DNA; note = miR4416-3p STTM
  misc_feature 12..14: note = CTA bulge
cctaggtgagactagcgacccgta

SEQ ID NO: 7 - DNA, 21 bp, synthetic construct
  source 1..21: mol_type = other DNA; note = miR4416-3p STTM with deletion
  misc_feature 11..12: note = location of deletion as compared to wild-type sequence
cctaggtgagagcgacccgta

SEQ ID NO: 8 - RNA, 21 nt, Glycine max
  source 1..21: mol_type = other RNA; note = miR4416-5p miRNA
tgggtgagagaaacgcgtatc

SEQ ID NO: 9 - RNA, 21 nt, Glycine max
  source 1..21: mol_type = other RNA; note = miR4416-3p miRNA
tacgggtcgctctcacctagg

SEQ ID NO: 10 - RNA, 135 nt, Glycine max
  source 1..135: mol_type = genomic RNA; note = miR4416-5p and miR4416-3p miRNA
gtcatctttgatctgggtgagagaaacgcgtatcgatggattgggttcagttctggtctcacacggtttgttctaacaatttgtactgactgtgttttgatcgatacgggtcgctctcacctaggccagagttgc

SEQ ID NO: 11 - RNA, 20 nt, Glycine max
  source 1..20: mol_type = mRNA; note = Le3 transcript
gataccgtttctttcacctt

SEQ ID NO: 12 - DNA, 48 bp, synthetic construct
  source 1..48: mol_type = other DNA; note = spacer
taagaagaagaaatctggtataaatttaatctggtattgttgttgttg

SEQ ID NO: 13 - DNA, 21 bp, Glycine max
  source 1..21: mol_type = genomic DNA; note = miR4416-5p
tgggtgagagaaacgcgtatc

SEQ ID NO: 14 - DNA, 21 bp, Glycine max
  source 1..21: mol_type = genomic DNA; note = miR4416-3p
tacgggtcgctctcacctagg

SEQ ID NO: 15 - protein, 29 aa, Glycine max
  source 1..29: note = sequence encoded by Le3 gene
KAHSTDTVSFTFNKFNPVQPNIMLQKDAS

SEQ ID NO: 16 - DNA, 86 bp, Glycine max
  source 1..86: mol_type = genomic DNA; note = Le3 gene
aaggcacactcgaccgataccgtttctttcaccttcaacaagttcaacccagtcaaccaaacattatgctccaaaaagatgctagt

SEQ ID NO: 17 - protein, 27 aa, synthetic construct
  source 1..27: note = protein encoded by Le3 mutated coding region Le3CR-1
KALDRYRFFHLQQVQPSPTKHYAPKRC

SEQ ID NO: 18 - DNA, 84 bp, synthetic construct
  source 1..84: mol_type = other DNA; note = Le3 mutated coding region Le3CR-1
aaggcactcgaccgataccgtttctttcaccttcaacaagttcaacccagtccaaccaaacattatgctccaaaaagatgctag

SEQ ID NO: 19 - protein, 25 aa, synthetic construct
  source 1..25: note = protein encoded by Le3 mutated coding region Le3CR-2
KDRYRFFHLQQVQPSPTKHYAPKRC

SEQ ID NO: 20 - DNA, 78 bp, synthetic construct
  source 1..78: mol_type = other DNA; note = Le3CR-2 mutated coding region of Le3
aaggaccgataccgtttctttcaccttcaacaagttcaacccagtccaaccaaacattatgctccaaaaagatcctag

SEQ ID NO: 21 - DNA, 16 bp, synthetic construct
  source 1..16: mol_type = other DNA; note = Universal reverse primer
gtgcagggtccgaggt

SEQ ID NO: 22 - DNA, 50 bp, synthetic construct
  source 1..50: mol_type = other DNA; note = miR4416-5p RT primer
gtcgtatccagtgcagggtccgaggtattcgcactggatacgacgatacg

SEQ ID NO: 23 - DNA, 21 bp, synthetic construct
  source 1..21: mol_type = other DNA; note = miR4416-5p F primer
cgcgtgtgggtgagagaaacg

SEQ ID NO: 24 - DNA, 50 bp, synthetic construct
  source 1..50: mol_type = other DNA; note = miR4416-3p RT primer
gtcgtatccagtgcagggtccgaggtattcgcactggatacgaccctagg

SEQ ID NO: 25 - DNA, 22 bp, synthetic construct
  source 1..22: mol_type = other DNA; note = miR4416-4p F primer
tcgcgtgtacgggtcgctctca

SEQ ID NO: 26 - DNA, 19 bp, synthetic construct
  source 1..19: mol_type = other DNA; note = GmELF1b qRT F
gttgaaaagccaggggaca

SEQ ID NO: 27 - DNA, 19 bp, synthetic construct
  source 1..19: mol_type = other DNA; note = GmELF1b qRT R
tcttaccccttgagcgtgg

SEQ ID NO: 28 - DNA, 22 bp, synthetic construct
  source 1..22: mol_type = other DNA; note = Le3 qRT F
tccgttgaaacccatgatgtga

SEQ ID NO: 29 - DNA, 19 bp, synthetic construct
  source 1..19: mol_type = other DNA; note = Le3 qRT R
tcttaccccttgagcgtgg

SEQ ID NO: 30 - DNA, 23 bp, synthetic construct
  source 1..23: mol_type = other DNA; note = PrecursorCh 03 qRT F
cagggcttactagttcacacagt

SEQ ID NO: 31 - DNA, 23 bp, synthetic construct
  source 1..23: mol_type = other DNA; note = PrecursorCh 03 qRT R
gacccgtatcgatcaaaacacac

SEQ ID NO: 32 - DNA, 24 bp, synthetic construct
  source 1..24: mol_type = other DNA; note = PrecursorCh 19 qRT F
acatgaaagttgtaaaagcacccc

SEQ ID NO: 33 - DNA, 22 bp, synthetic construct
  source 1..22: mol_type = other DNA; note = PrecursorCh 19 qRT R
ttgcagcctagcaaaacttgtc

SEQ ID NO: 34 - DNA, 100 bp, synthetic construct
  source 1..100: mol_type = other DNA; note = miR4416-5p STTM Forward
ccgggatacgcgtttctactctcacccagttgttgttgttatggtctaatttaaatatggtctaaagaagaagaatgatacgcgtttctactctcaccca

SEQ ID NO: 35 - DNA, 100 bp, synthetic construct
  source 1..100: mol_type = other DNA; note = miR4416-5p STTM Reverse
gatctgggtgagagtagaaacgcgtatcattcttcttctttagaccatatttaaattagaccataacaacaacaactgggtgagagtagaaacgcgtatc

SEQ ID NO: 36 - DNA, 100 bp, synthetic construct
  source 1..100: mol_type = other DNA; note = miR4416-3p STTM Forward
ccggcctaggtgagactagcgacccgtagttgttgttgttatggtctaatttaaatatggtctaaagaagaagaatcctaggtgagactagcgacccgta

SEQ ID NO: 37 - DNA, 100 bp, synthetic construct
  source 1..100: mol_type = other DNA; note = miR4416-3p STTM Reverse
gatctacgggtcgctagtctcacctaggattcttcttctttagaccatatttaaattagaccataacaacaacaactacgggtcgctagtctcacctagg

SEQ ID NO: 38 - DNA, 25 bp, synthetic construct
  source 1..25: mol_type = other DNA; note = Le3 Reverse GSP1
ccggtggtggcagagaacccaacgc

SEQ ID NO: 39 - DNA, 26 bp, synthetic construct
  source 1..26: mol_type = other DNA; note = Le3 Reverse GSP2
acccccacgatgccgttttgacggac

SEQ ID NO: 40 - DNA, 25 bp, synthetic construct
  source 1..25: mol_type = other DNA; note = Le3 Forward GSP1
caaagttggcagcaacggcgtgccc

SEQ ID NO: 41 - DNA, 25 bp, synthetic construct
  source 1..25: mol_type = other DNA; note = Le3 Forward GSP2
tcggtcgtgccctttacgctgcccc

SEQ ID NO: 42 - DNA, 26 bp, synthetic construct
  source 1..26: mol_type = other DNA; note = PrecursorCh 03 Reverse GSP1
actctagtctaggtgagaggacccgt

SEQ ID NO: 43 - DNA, 28 bp, synthetic construct
  source 1..28: mol_type = other DNA; note = PrecursorCh 03 Reverse GSP2
ggacccgtatcgatcaaaacacactcac

SEQ ID NO: 44 - DNA, 25 bp, synthetic construct
  source 1..25: mol_type = other DNA; note = PrecursorCh 03 Forward GSP
acgcgtatcgatgagattcagggct

SEQ ID NO: 45 - DNA, 25 bp, synthetic construct
  source 1..25: mol_type = other DNA; note = PrecursorCh 19 Reverse GSP1
ccaccactcttctccaattcgggct

SEQ ID NO: 46 - DNA, 25 bp, synthetic construct
  source 1..25: mol_type = other DNA; note = PrecursorCh 19 Reverse GSP2
tctggcctaggtgagagcgacccgt

SEQ ID NO: 47 - DNA, 26 bp, synthetic construct
  source 1..26: mol_type = other DNA; note = PrecursorCh 19 Forward GSP1
acgcgtatcgatggattgggttcagt

SEQ ID NO: 48 - DNA, 26 bp, synthetic construct
  source 1..26: mol_type = other DNA; note = PrecursorCh 19 Forward GSP2
tgggttcagttctggtctcacacggt

SEQ ID NO: 49 - DNA, 25 bp, synthetic construct
  source 1..25: mol_type = other DNA; note = Le3_pGES201_F
ggattggtatcggtcgagtgtgcct

SEQ ID NO: 50 - DNA, 25 bp, synthetic construct
  source 1..25: mol_type = other DNA; note = Le3_pGES201_R
aaacaggcacactcgaccgatacca

SEQ ID NO: 51 - DNA, 40 bp, synthetic construct
  source 1..40: mol_type = other DNA; note = Le3_CDS_F
caccatttacgaacgatagcatggccacctccaacttctc

SEQ ID NO: 52 - DNA, 41 bp, synthetic construct
  source 1..41: mol_type = other DNA; note = Le3_CDS_R
actggtgatttttgcggactttagatggcctcattgagcac

SEQ ID NO: 53 - DNA, 163 bp, synthetic construct
  source 1..163: mol_type = other DNA; note = miR4416-5p F primer
ccggttaacagtcgttatttgcggtgggtgagagaaacgcgtatccatgcaagcgcaggtggtgggtgggacttgatgcaatctaagtgctgtgccagccaagccataggtcttttggaactggatacgcgtttctctcacccacagccataaacgacttcac

SEQ ID NO: 54 - DNA, 163 bp, synthetic construct
  source 1..163: mol_type = other DNA; note = miR4416-5p R primer
gatcgtgaagtcgtttatggctgtgggtgagagaaacgcgtatccagttccaaaagacctatggcttggctggcacagcacttagattgcatcaagtcccacccaccacctgcgcttgcatggatacgcgtttctctcacccaccgcaaataacgactgttaa

SEQ ID NO: 55 - DNA, 36 bp, synthetic construct
  source 1..36: mol_type = other DNA; note = Le3 NSL F
ttctgcccaaattcgcgcatggccacctccaacttc

SEQ ID NO: 56 - DNA, 57 bp, synthetic construct
  source 1..57: mol_type = other DNA; note = Le3 NSL R
ctcctcgcccttgctcactccacctcctccaccacctccgatggcctcattgagcac

SEQ ID NO: 57 - DNA, 131 bp, synthetic construct
  source 1..131: mol_type = other DNA; note = pGES401 backbone and guide RNA sequences
cctatggtcatctttgatctgggtgagagaaacgcgtatcgatggattgggttcagttctggtctcacacggtttgttctaacaatttgtactgactgtgttttgatcgatacgggtcgctctcacctagg

SEQ ID NO: 58 - DNA, 46 bp, synthetic construct
  source 1..46: mol_type = other DNA; note = pGES401 backbone and guide RNA for Mutant 1
cctatggtcatctttgatctcggtgagagaaacgcgtatacctagg

SEQ ID NO: 59 - DNA, 37 bp, synthetic construct
  source 1..37: mol_type = other DNA; note = pGES401 backbone and guide RNA for Mutant 2
cctatggtcatctttgatctgggtgagagaacctagg

SEQ ID NO: 60 - DNA, 99 bp, synthetic construct
  source 1..99: mol_type = other DNA; note = pGES401 backbone and guide RNA for Mutant 3
cctatggcgatggattgggttcagttctggtctcacacggtttgttctaacaatttgtactgactgtgttttgatcgatacgggtcgctctcacctagg

SEQ ID NO: 61 - DNA, 41 bp, synthetic construct
  source 1..41: mol_type = other DNA; note = pGES401 backbone and guide RNA for Mutant 4
cctatggtcatctttgatctgggtgagagaaacgccctagg

SEQ ID NO: 62 - DNA, 45 bp, synthetic construct
  source 1..45: mol_type = other DNA; note = pGES401 backbone and guide RNA for Mutant 5
cctatggtcatctttgatctgggtgagagaaacgcgtatcctagg

SEQ ID NO: 63 - DNA, 35 bp, synthetic construct
  source 1..35: mol_type = other DNA; note = pGES401 backbone and guide RNA for Mutant 6
cctatggtcatctttgatctgggtgagagaaaagg

SEQ ID NO: 64 - DNA, 131 bp, synthetic construct; identical to SEQ ID NO: 57
  source 1..131: mol_type = other DNA; note = pGES401 backbone and guide RNA for Mutant 7

(19) United States
(12) Patent Application Publication
(10) Pub. No.: US 2026/0297600 A1
(43) Pub. Date: Oct. 1, 2026

(54) PLANT GROWTH AND SEED YIELD ENHANCEMENT METHODS AND CONSTRUCTS

(71) Applicant: Purdue Research Foundation, West Lafayette, IN (US)

(72) Inventors: Jianxin Ma, West Lafayette, IN (US); Jingbo Duan, West Lafayette, IN (US)

(21) Appl. No.: 19/490,931

(22) PCT Filed: Jun. 7, 2024
(86) PCT No.: PCT/US2024/033039

§ 371 Date: Dec. 8, 2025

Related U.S. Application Data
(60) Provisional application No. 63/471,685, filed on Jun. 7, 2023.

(51) Int. Cl. C12N 15/82; C12N 9/22; C12N 15/11

(52) U.S. Cl. CPC C12N 15/8218; C12N 9/226; C12N 15/11; C12N 15/8213; C12N 15/8261; C12N 2310/20

(57) ABSTRACT

Expression constructs for modulating expression of Le3 and/or miR4416-5p or miR4416-3p, genome-editing systems for leveraging the same to enhance plant yield, plant growth, and seed composition. Generally improved transgenic plants leveraging such constructs are also provided, as are methods for enhancing plant growth using such expression constructs and/or genome-editing systems.

### PRIORITY

This patent application is related to, claims the priority benefit of U.S. Provisional Patent Application No. 63/471,685 filed Jun. 7, 2023. The content of the foregoing application is hereby incorporated by reference in its entirety into this disclosure.

### GOVERNMENT SUPPORT

This invention was made with government support under 2006668 and 2128023 awarded by the National Science Foundation (NSF). The government has certain rights in the invention.

### TECHNICAL FIELD

The present disclosure relates to enhancing nodulation, plant growth, and seed yield in legumes and, in particular, soybean cultivars, through targeted genetic mutations and modifications, including methods and compositions for making such mutations and modifications. manipulation of lectin gene Le3, expression.

### SEQUENCE LISTINGS

The sequences herein (SEQ ID NOS: 1-64) are also provided in computer readable form encoded in a file filed herewith and incorporated herein by reference, which was created on Jun. 7, 2024, named 70176-02_SequenceListing_07JUN2024.xml, and is 72,673 bytes in size. The information recorded in computer readable form is identical to the written Sequence Listings provided below, pursuant to 37 C.F.R. § 1.821(f).

### BACKGROUND

This section introduces aspects that may help facilitate a better understanding of the disclosure. Accordingly, these statements are to be read in this light and are not to be understood as admissions about what is or is not prior art.

Legumes are plants, such as soybeans, alfalfa, clover, peas, beans, lentils, lupins, mesquite, and peanuts, that form a symbiotic relationship between their roots and bacteria, specifically of the family Rhizobiaceae. Rhizobial lipo-chitooligosaccharidic nodulation (Nod) factors are the key signal molecules responsible for induction of plant responses that lead to nodule formulation. Upon perception of plant flavonoids, Rhizobia secrete Nod factors, which induce root hair curling around the bacteria and the subsequent development of infection threads that allow the bacteria to penetrate the cortical cells of the roots to form nodules.

Nodulation in legumes provides a major conduit of available nitrogen into the biosphere. More specifically, the plant provides the bacteria both sustenance and an energy source in the form of adenosine triphosphate (ATP) that is generated by photosynthesis. In return, the bacteria fix elemental nitrogen from the atmosphere into ammonia, a usable form of nitrogen that is digestible by plants. This interaction, called symbiotic nitrogen fixation (SNF), is a critical biological process for agricultural and natural ecosystems because it enables the plant hosts to access atmospheric nitrogen and, thus, provides a rich nitrogen source to the plant (e.g., approximately 50-60% of the nitrogen required for legume plant growth). Zahran, Rihizobium-legume symbiosis and nitrogen fixation under severe conditions and in an arid climate, Microbiology & Molecular Biology Reviews 63:968-989 (1999).

SNF occurs in specialized root organs known as nodules of legumes and is a major source of nitrogen in agricultural and natural eco-systems. As nitrogen is the nutrient that most frequently limits the growth of green plants, optimizing its application is a key to optimizing plant yield.

As SNF by rhizobia within the nodules is an energy-intensive process, legumes have evolved a strategy referred to as autoregulation of nodulation (AON) to systemically control nodule numbers for a cost-benefit balance. Ferguson et al., Legume nodulation: The host controls the party, Plant Cell & Environment 42:41-51 (2019). AON is a negative-feedback mechanism used by legumes to restrict root nodule numbers so as to balance symbiosis and plant growth. Disruption of AON in legumes usually leads to supernodulation, which can reduce plant productivity. In both Lotus japonicus and soybean, AON involves a mobile microRNA (miRNA), miR2111, which can enhance nodulation through posttranscriptional regulation of the symbiosis suppressor gene, TOO MUCH LOVE (TML), that encodes a Kelch repeat-containing F-box protein in roots. Tsikou et al., Systemic control of legume susceptibility to rhizobial infection by a mobile microRNA, Science 362:233-236 (2018); Zhang et al., Shoot-derived miR2111 controls legume root and nodule development, Plant Cell & Environment 44:1627-1641 (2021). As shown in typical AON-defective mutants, tml knockouts can display hypernodulation with stunted root growth. Magori et al., TOO MUCH LOVE, a root regulator associated with the long-distance control of nodulation in Lotus japonicus, Molecular Plant-Microbe Interactions 22:259-268 (2009). Despite the conservation of this strategy, symbiosis exhibits extensive species-specificity that involves a variety of different signaling molecules. Ren et al., Rhizobial tRNA-derived small RNAs are signal molecules regulating plant nodulation, Science 365:919-922 (2019).

Legumes are generally higher in protein content than other plant families due to the availability of nitrogen from nitrogen fixation. The high protein content makes legumes one of the most important food crops for both human consumption and animal feed. Further, legumes are used in crop rotation practice to increase the nitrogen content of soils (e.g., through SNF) for future growth seasons and to reduce the amount of fertilizer required. This has cost-benefits to the grower and can reduce nitrogen runoff.

### SUMMARY

A leguminous plant is provided that comprises at least one mutation. The at least one mutation can comprise a first mutation that upregulates expression of a Le3 gene in the leguminous plant as compared to expression in a corresponding wild-type leguminous plant; and/or a second mutation that downregulates or silences expression of microRNA (miRNA) 4416-5p or miR4416-3p. The plant is a soybean cultivar. In certain embodiments, the plant comprises a soybean. In certain embodiments, the plant comprises Glycine max. The plant can be a plant, a plant cell, or a plant material (i.e., portion of a plant such as a leaf, a stem, a root, etc.). The at least one mutation can result in interference with a miRNA-Le3 interaction in the plant.

Upregulation can be achieved by the incorporation of one or more exogenous Le3 genes into a genome of the plant. The one or more exogenous Le3 genes can be, for example, integrated at a single genomic locus or at multiple genomic loci. In certain embodiments, upregulation can be achieved or facilitated by downregulating or silencing expression of miRNA 4416-5p or miR4416-3p, for example. The Le3 gene can be operably linked to a promoter that upregulates expression of the Le3 gene. In certain embodiments, upregulation is facilitated by the incorporation of one or more gene regulatory elements (e.g., into the plant genome).

In certain embodiments, the at least one mutation comprises both the first mutation and the second mutation, the second mutation is encoded by SEQ ID NO: 3 or SEQ ID NO: 4. The second mutation can be encoded by one or more modified miR4416-5p sequences or one or more modified miR4416-3p sequences Optionally, the modified miR4416-5p sequences each independently comprise a miR4416-5p short tandem target mimic (STTM) sequence or a sequence of wild-type miR4416-5p comprising an insertion or deletion therein, and/or the modified miR4416-3p sequences each comprise a miR4416-3p STTM sequence or a sequence of wild-type miR4416-3p comprising an insertion or deletion therein. The modified miR4416-5p sequence can comprise a wild-type miR4416-5p sequence with a bulge sequence inserted therein. The modified miR4416-3p sequence can comprise a wild-type miR4416-3p sequence with a bulge sequence inserted therein. The bulge sequence can be or comprise CTA.

In certain embodiments, the second mutation is encoded by at least two modified miR4416-5p sequences and/or at least two modified miR4416-3p sequences. Each of the modified miR4416-5p or miR4416-3p sequences can be separated by a spacer. In certain embodiments, each of the at least two modified miR4416-5p sequences independently comprises SEQ ID NO: 3 or SEQ ID NO: 4, or a functional variant of SEQ ID NO: 3 or SEQ ID NO: 4 (e.g., to the extent the functional variant is not wild-type miR4416-5p); and/or each of the at least two modified miR4416-3p sequences independently comprises SEQ ID NO: 6 or SEQ ID NO: 7, or a functional variant of SEQ ID NO: 6 or SEQ ID NO: 7 (e.g., to the extent the functional variant is not wild-type miR4416-3p). The spacer can be or comprise a length sufficient to allow for binding between two of the modified miR4416-5p sequences or two of the modified miR4416-3p sequences in a loop structure. The spacer can be or comprise between about 3 to about 99 nucleic acids and is optionally about 48 nucleic acids.

In certain embodiments, the at least one mutation is encoded by SEQ ID NO: 1 or a functional variant of SEQ ID NO: 1.

In one embodiment, the present disclosure encompasses an isolated nucleic acid construct which is useful for transforming target plant cells. The present invention provides for where the target cells are plant root cells. In another embodiment the target plant cells can be any other plant cells which can accept transfer of the isolated nucleic acid.

In certain embodiments, an isolated nucleic acid construct is provided that comprises a nucleic acid sequence of amiR4416-5p* and a nucleic acid sequence of amiR4416-5p, with a spacer segment of nucleic acids positioned therebetween. The spacer segment can have, for example, enough base pairs to allow for binding between the amiR4416-5p* and the nucleic acid sequence of amiR4416-5p nucleic acid sequences.

Isolated nucleic acid constructs are also provided. In certain embodiments, an isolated nucleic acid construct comprises the nucleic acid sequence of amiR4416-5p* and the nucleic acid sequence of amiR4416-5p, where the construct forms a self-binding loop structure as shown in FIG. 6A. The isolated nucleic acid construct can comprise the nucleic acid sequence shown in FIG. 6B (SEQ ID NO: 1). In certain embodiments, the isolated nucleic acid construct comprises a nucleic acid sequence of miR4416-5p which has been interrupted by a “bulge sequence” insert, or the nucleic acid sequence of miR4416-5p ST™.

In certain embodiments, the isolated nucleic acid construct comprises the nucleic acid sequence of miR4416-5p with an insertion that interrupts the sequence, and/or the nucleic acid sequence of miR4416-5p STTM (FIG. 7A), where, optionally, the construct further comprises a spacer nucleic acid sequence having a non-coding and non-hybridizing sequence of about 48 nucleic acids (such as 48 nucleic acids). Such non-coding/non-hybridizing spacer can be from about 6 to about 96 nucleic acids (such as about 6 to 96 nucleic acids, 6 to 96 nucleic acids, or about 6 to about 96 nucleic acids) in length. Such non-coding/non-hybridizing spacer can be about 3 to about 99 nucleic acids (such as about 3 to 99 nucleic acids, 3 to 99 nucleic acids, or about 3 to about 99 nucleic acids) in length. The non-coding/non-hybridizing spacer can be about 3 to about 60 nucleic acids (such as about 3 to 60 nucleic acids, 3 to 60 nucleic acids, or about 3 to about 60 nucleic acids) in length. Thus, the nucleic acid construct can comprise more than one copy of an interrupted miR4416-5p nucleic acid sequence.

The isolated nucleic construct can comprise the nucleic acid sequence of FIG. 7B (SEQ ID NO: 2). The isolated nucleic construct can have the nucleic acid sequence of FIG. 7B (SEQ ID NO: 2). The isolated nucleic acid can consist of the nucleic acid sequence as shown in FIG. 7B (SEQ ID NO: 2). The isolated nucleic acid construct can comprise the nucleic acid sequence of miR4416-3p interrupted by a “bulge sequence” and/or the nucleic acid sequence of miR4416-3p short tandem target mimic (STTM).

In certain embodiments, the isolated nucleic acid construct comprises the nucleic acid sequence of miR4416-3p, interrupted by an insertion, and/or the nucleic acid sequence of miR4416-3p ST™, where the construct further comprises a spacer nucleic acid sequence having a non-coding and non-hybridizing sequence of at or about 48 nucleic acids between more than one copy of the miR4416-3p ST™. In certain embodiments, the spacer is from about 6 to about 96 nucleic acids (such as about 6 to 96 nucleic acids, 6 to 96 nucleic acids, or about 6 to about 96 nucleic acids) in length. The spacer can be about 3 to about 99 nucleic acids (such as about 3 to 99 nucleic acids, 3 to 99 nucleic acids, or about 3 to about 99 nucleic acids) in length. The spacer can be about 3 to about 60 nucleic acids (such as about 3 to 60 nucleic acids, 3 to 60 nucleic acids, or about 3 to about 60 nucleic acids) in length.

In certain embodiments, the isolated nucleic construct comprises more than one copy of an interrupted miR4416-3p nucleic acid sequence. In certain embodiments, the isolated nucleic construct has or comprises the nucleic acid sequence of FIG. 9B (SEQ ID NO: 5). In certain embodiments, the isolated nucleic acid consists of the nucleic acid sequence as shown in FIG. 9B (SEQ ID NO: 5). The isolated nucleic acid construct can comprise (or consist of) the sequence shown in FIG. 9C (SEQ ID NO: 6). The isolated nucleic acid construct can comprise (or consist of) the sequence shown in FIG. 9D (SEQ ID NO: 7).

In certain embodiments, an expression construct is provided that comprises a first nucleic acid sequence that upregulates expression of a Le3 gene in a leguminous plant as compared to wild-type expression in a corresponding wild-type leguminous plant; and/or a second nucleic acid sequence that downregulates or silences expression of miR4416-5p or miR4416-3p. The expression construct can comprise both the first nucleic acid sequence and the second nucleic acid sequence. In certain embodiments, the expression construct comprises SEQ ID NO: 1 or SEQ ID NO: 2.

The expression construct can comprise the first nucleic acid sequence. The first nucleic acid sequence can encode two or more Le3 genes. The two or more Le3 genes can be encoded at a single genomic locus or at multiple genomic loci. In certain embodiments, the first nucleic acid sequence comprises one or more gene regulatory elements. The one or more gene regulatory elements can comprise at least a promoter for upregulating expression of the Le3 gene (i.e., as compared to wild-type expression of the Le3 gene).

The expression construct can comprise the second nucleic acid sequence. The second nucleic acid sequence can comprise SEQ ID NO: 3 or SEQ ID NO: 4, or a functional variant of SEQ ID NO: 3 or SEQ ID NO: 4 (e.g., to the extent the functional variant is not wild-type miR4416-5p). The second nucleic acid sequence can comprise one or more modified miR4416-5p sequences or one or more modified miR4416-3p sequences. In certain embodiments, at least one of the modified miR4416-5p sequences comprises a miR4416-5p STTM sequence or a sequence of wild-type miR4416-5p comprising an insertion or deletion therein. In certain embodiments, at least one of the modified miR4416-3p sequences comprises a miR4416-3p STTM sequence or a sequence of wild-type miR4416-3p comprising an insertion or deletion therein. The one or more modified miR4416-5p sequences can comprise a wild-type miR4416-5p sequence with a bulge sequence inserted therein. The one or more modified miR4416-3p sequences can comprise a wild-type miR4416-3p sequence with a bulge sequence inserted therein. The bulge sequence can be or comprise CTA, for example.

The expression construct can encode a spacer. For example, the second nucleic acid sequence can, in certain embodiments, encode two or more modified miR4416-5p or two or more modified miR4416-3p, wherein each of the modified miR4416-5p or miR4416-3p sequences is separated by a spacer. The spacer can be of a length sufficient to allow for binding between two of the modified miR4416-5p sequences or two of the modified miR4416-3p sequences in a loop structure. The spacer can comprise between about 3 to about 99 nucleic acids. In certain embodiments, the spacer is about 48 nucleic acids.

Methods for enhancing plant growth are also provided. In certain embodiments, a method for enhancing plant growth comprises inoculating a plant cell with an isolated nucleic acid construct which comprises the nucleic acid sequence of amiR4416-5p* and the nucleic acid sequence of amiR4416-5p, where the construct forms a self-binding, or hairpin, loop structure as shown in FIG. 6A in which amiR4416-5p* and amiR4416-5p form the stem. The present disclosure provides for methods for enhancing plant growth, comprising transforming a plant cell with the construct comprising an isolated nucleic acid construct which comprises the nucleic acid sequence of amiR4416-3p* and the nucleic acid sequence of amiR4416-3p, where the construct forms a hairpin loop structure as shown in FIG. 6A.

In certain embodiments, a method for enhancing plant growth comprises transforming a plant cell with an isolated nucleic acid construct which comprises the nucleic acid sequence of miR4416-5p STTM (SEQ ID NO: 3) and the nucleic acid sequence of miR4416-5p that has been interrupted or otherwise disrupted (e.g., SEQ ID NO: 4). The methods for enhancing plant growth can comprise transforming a plant cell with an isolated nucleic acid construct comprising the nucleic acid sequence of miR4416-3p STTM (SEQ ID NO: 6). Accordingly, the methods can comprise using a construct comprising the nucleic acid sequence of SEQ ID NO: 7 which is a miR4416-3p sequence that has been interrupted or otherwise disrupted.

In certain embodiments of methods for enhancing plant growth, the plant is a soybean plant. In certain embodiments, the introduction of the nucleic acid construct to the plant cell is done prior to planting the plant. In certain embodiments, the introduction of the nucleic acid construct to the plant cell is done after planting the plant.

Still further, methods of using nucleic acid constructs for transforming target plant cells with useful transformational modifications are provided. Such methods can result in improved plant growth and seed yield. Such modifications can include transformation with the particular constructs hereof, or variations thereof that are in accordance with the spirit of the disclosure.

In certain embodiments, methods for transforming plant cells and plants to create stable varieties which demonstrate better than wild-type growth characteristics are provided. In certain embodiments, the plants, plant cells, roots and the subsequent seeds and plants that grow as a result of the method for transforming plant cells are provided.

Methods of enhancing plant growth by modulating or upregulating the expression of the Le3 gene are also provided. This is a previously unknown function of the Le3 gene. As follows, the use of nucleic acid constructs which interfere with the operation of miRNAs and, thus, that interfere with the expression of native Le3 gene is also provided. By disrupting the interfering miRNAs, the methods, uses, and constructs can modulate the Le3 gene expression and effectively allow for more productive Le3 expression to occur (e.g., as compared to expression in wild-type plants). At least one effect of this Le3 gene activity on a plant can be improved health, growth, productivity, and development.

In certain embodiments, a method for enhancing leguminous plant growth comprises upregulating expression of a Le3 gene in a plant as compared to expression in a corresponding wild-type leguminous plant; and/or downregulating or silencing expression of miR4416-5p, miR4416-3p, or both miR4416-5p and miR4416-3p in the plant as compared to expression in a corresponding wild-type leguminous plant. The method can comprise, for example, both upregulating expression of a Le3 gene and downregulating or silencing expression of miR4416-5p or miR4416-3p. The plant can be a whole plant, a plant cell, or any portion of a plant. The plant can be a leguminous plant. The plant can be a soybean plant or a soybean cultivar. The plant can comprise Glycine max.

Upregulating expression of a Le3 gene and/or downregulating or silencing expression of miR4416-5p and/or miR4416-3p can comprise introducing one or more expression constructs (e.g., any described herein or otherwise contemplated hereby) into the plant or incorporating modifications into the sequence of miR4416-5p and/or the sequence of miR4416-3p of the plant. Introducing or incorporating can be performed by gene editing techniques such as, optionally, CRISPR-Cas9, TALENs, or homologous recombination. Introducing or incorporating can be performed prior to planting the plant or after planting the plant.

Upregulating expression of a Le3 gene can comprise incorporating one or more exogenous Le3 genes into a genome of the plant. The one or more exogenous Le3 genes can be integrated at a single genomic locus. The one or more exogenous Le3 genes can comprise at least two exogenous Le3 genes that are integrated at multiple genomic loci. The Le3 gene can be operably linked to a promoter that upregulates expression of the Le3 gene. In certain embodiments, upregulating comprises incorporating one or more gene regulatory elements into a genome of the plant.

In certain embodiments, downregulating or silencing expression of miR4416-5p and/or miR4416-3p comprises transforming the plant with a construct comprising SEQ ID NO: 3, SEQ ID NO: 4, SEQ ID NO: 6, SEQ ID NO: 7, or two or more of SEQ ID NOS: 3-7 (or a functional variant of any of SEQ ID NOS: 3-7 to the extent such variants are not the corresponding wild-type sequence). Downregulating or silencing expression of miR4416-5p and/or miR4416-3p can comprise, for example, transforming the plant with an expression construct comprising one or more modified miR4416-5p sequences or one or more modified miR4416-3p sequences. In certain embodiments, at least one of the modified miR4416-5p sequences comprises a miR4416-5p STTM sequence or a sequence of wild-type miR4416-5p comprising an insertion or deletion therein. In certain embodiments, at least one of the modified miR4416-3p sequences comprises a miR4416-3p STTM sequence or a sequence of wild-type miR4416-3p comprising an insertion or deletion therein. The one or more modified miR4416-5p sequence can comprise a wild-type miR4416-5p sequence with a bulge sequence inserted therein. The one or more modified miR4416-3p sequence can comprise a wild-type miR4416-3p sequence with a bulge sequence inserted therein. The bulge sequence can be or comprise CTA, for example.

In certain embodiments, incorporating comprises incorporating an insertion into a sequence of miR4416-5p or miR4416-3p of the plant. In certain embodiments, introducing one or more expression constructs into the plant further comprises transforming the leguminous plant with one or more of expression constructs (e.g., any of the expression constructs described herein or otherwise contemplated).

Plant yield can be enhanced by performing the method by at or more than about 20% as compared to plant yield of a corresponding wild-type plant. In certain embodiments, performance of the method results in an increase in seed protein concentration as compared to seed protein concentration in a corresponding wild-type plant. In certain embodiments, the method of enhancing plant growth further comprises inoculating, or having inoculated, the modified plant with at least one rhizobium to initiate nodulation.

Genetically modified organism (GMO) plant cells are also provided. In certain embodiments, such GMO plant cells incorporate upregulating the Le3 gene activity in a plant cell to foster enhanced properties (e.g., growth, seed development, improved health and/or productivity). Methods for upregulating by means of transforming a plant cell with an additional nucleic acid construct which encodes for an exogenous Le3 gene are also provided. In certain embodiments, the method can comprise introducing one or more additional copies of the Le3 gene into the plant cell genetic material. Methods for upregulating a gene are well known in the art. For example, an additional promoter can be introduced into the genome operably linked to the Le3 gene. Regulatory elements such as enhancers and methods for such manipulation are known in the art. Schmitz et al., Cis-regulatory sequences in plants: Their importance, discovery, and future challenges, The Plant Cell 34:718-741 (2022). Accordingly, in certain embodiments, a plant cell which has been modified to allow for Le3 expression to be uninhibited is provided, such modification comprising, for example, interference with the miRNA-Le3 interaction. A plant cell hereof can be modified to enhance Le3 activity, by introducing regulatory elements which promote or otherwise upregulate the activity of the Le3 gene. Suitable regulatory elements are known in the art. Zhang et al., Isolation and characterization of “GmScream” promoters that regulate highly expressing soybean (Glycine max Merr.) genes, plant Science 241:189-198 (2015); Callis et al., Ubiquitin extension proteins of Arabidopsis thaliana. Structure, localization, and expression of their promotors in transgenic tobacco, J Biological Chemistry 265 (21): 12486-12493 (1990).

In certain embodiments, the plant cell hereof exhibits enhanced Le3 activity, wherein the plant cell has been transformed with a genetic construct that provides for exogenous Le3 gene activity. In certain embodiments, one or more additional copies of the Le3 gene is introduced into the genetic material of a plant cell, wherein the one or more additional copies are, optionally, operably linked to promotor elements which allow for expression in such transformed plant cell.

In certain embodiments, a plant cell is provided which contains a nucleic acid construct comprising the nucleic acid sequence of miR4416-5p ST™. In certain embodiments, the plant cell contains a nucleic acid construct comprising the nucleic acid sequence of miR4416-3p ST™.

A “bulge sequence” in the nucleic acid sequences of miR4416-5p STTM and miR4416-3p STTM is also provided, for example, as diagramed in FIGS. 7A and 9A, respectively, as 3 nucleic acids. In particular, the “bulge sequence” is CTA. The “bulge sequence” can be, for example, from about 1 to 9 nucleic acids (such as about 1 to about 9 nucleic acids, 1 to about 9 nucleic acids, or 1-9 nucleic acids).

The plant cell hereof can contain an isolated nucleic acid construct which comprises the nucleic acid sequence of miR4416-5p STTM (SEQ ID NO: 3), which comprises a miR4416-5p sequence that further comprises an insert (e.g., CTA). The plant cell can contain an isolated nucleic acid construct which comprises the nucleic acid sequence of SEQ ID NO: 4, which is a miR4416-5p which has been interrupted. The plant cell can contain an isolated nucleic acid construct which comprises the nucleic acid sequence of SEQ ID NO: 4, each of which is a miR4416-5p sequence that has been interrupted (i.e., comprises an insertion or deletion) such that the resulting sequence is not entirely complementary to the miR4416-5p wild-type sequence.

In certain embodiments, a plant cell is provided, such plant cell comprising a construct comprising an isolated nucleic acid construct which comprises the nucleic acid sequence of miR4416-3p STTM (SEQ ID NO: 6). Further provided is a method for using a construct comprising the nucleic acid sequence of SEQ ID NO: 7, which is a miR4416-3p sequence that has been interrupted (i.e., comprises an insertion or deletion). Thus, the plant cell can have or comprise a construct comprising the nucleic acid sequence of miR4416-3p, which has been interrupted or otherwise disrupted (SEQ ID NO: 7). In certain embodiments, the plant cell has a construct comprising the nucleic acid sequence of miR4416-3p which has been interrupted by an insert (SEQ ID NO: 6).

The plant cell hereof can, in certain embodiments, contain a nucleic acid construct comprising the nucleic acid sequence of amiR4415-5p* and amiR4416-5p. In certain embodiments, the plant cell comprises the nucleic acid construct comprising the nucleic acid sequence of amiR4415-3p* and amiR4416-3p.

A plant comprising a plant cell hereof is also provided. In certain embodiments, the plant comprises a plant cell containing a nucleic acid construct comprising the nucleic acid sequence of miR4416-5p which has been interrupted or otherwise disrupted (SEQ ID NO: 4). Also provided for is a plant where the nucleic acid sequence of miR4416-5p has been interrupted or otherwise disrupted, for example by a deletion or insertion of a space (SEQ ID NO: 4). In certain embodiments, the plant comprises a plant cell which contains a nucleic acid construct comprising the nucleic acid sequence of miR4416-3p which has been interrupted or otherwise disrupted (SEQ ID NO: 7). In certain embodiments, the plant comprises a cell having or comprising the nucleic acid sequence of miR4416-3p, which has been disrupted (SEQ ID NO: 7).

Use of the constructs alone or in tandem with each other is also provided. Thus, methods for enhancing plant growth are provided, such methods comprising transforming a plant cell with a construct comprising an isolated nucleic acid construct which comprises the nucleic acid sequence miR4416-5p which has been interrupted or disrupted (e.g., SEQ ID NO: 4), in combination with a construct comprising the nucleic acid sequence of miR4416-3p which has been interrupted or disrupted (e.g., SEQ ID NO: 7). Further, the present disclosure provides for a single nucleic acid construct which comprises one or more copies of both miR4416-5p (SEQ ID NO: 4) and miR4416-3p (SEQ ID NO: 7) which have been interrupted or disrupted. In general, the inhibition, disruption, or deletion of miR4416-5p (SEQ ID NO: 4) and/or miR4416-3p (SEQ ID NO: 7) activity enhances plant growth and seed production.

Still further, seeds and plants grown from plant cells that have been modified by the constructs and methods hereof are provided.

### DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIGS. 1A-1G show images, schematics and data related to miR4416-5p regulating Le3 post-transcriptionally. FIG. 1A shows a schematic of miR4416-5p (SEQ ID NO: 8) and miR4416-3p (SEQ ID NO: 9) (in total, SEQ ID NO: 10). FIG. 1B is an image of sample collection sites from different soybean tissues. FIGS. 1C-1E are graphs of expression levels/abundance measurements of MIR4416, miR4416-5p, and Le3 in different tissues as determined by quantitative reverse transcription polymerase chain reaction (qRT-PCR)/stem-loop real-time (RT)-qRT-PCR. The numbers below the x-axis in each figure denote the corresponding tissues shown in FIG. 1B. The shoot apexes values were set as “1” and the others adjusted accordingly in FIG. 1C and FIG. 1D. The roots value was set as “1” and the others adjusted accordingly in FIG. 1E. FIG. 1F shows the sequence of miR4416-5p (SEQ ID NO: 8), its putative target transcript Le3 (SEQ ID NO: 11) and the cleavage site and frequency (indicated by arrow and ratio) detected in the V1-stage uninoculated roots. FIG. 1G shows images of subcellular localization of Le3. The “YFP” panel (image shown in yellow (Y)) represents signals of Le3 fused eYFP; the “mCherry-AtPIP2A” panel represents signals of plasma membrane-targeted mCherry marker proteins; the “Merged” panel show merged YFP, mCherry and DIC signals.

FIGS. 2A-2I show images and data related to the modulation of soybean nodulation by miR4416-5p and its putative target, Le3. FIG. 2A is a graph of abundance data for miR4416-5p at early-stage rhizobial (USDA110) infection of soybean root hairs detected by stem-loop RT-qRT-PCR. FIG. 2B is a graph of the expression of Le3 measured by qRT-PCR in the same samples as described in FIG. 2A. Values in FIG. 2A and FIG. 2B, with one set as “1” and labeled with “N”, and the others adjusted accordingly, are shown as means±s.e. from three biological replicates; p-values were calculated using Student's t-test (*p≤0.05; **p≤0.01). FIGS. 2C and 2D are graphs of abundance and expression levels, respectively, of the miR4416-5p and Le3 in the miR4416-5p stable transgenic plants as determined by stem-loop RT-qRT-PCR/qRT-PCR. Wild-type (WT) values in FIG. 2C and FIG. 2D were set as “1” and the others adjusted accordingly, and are shown as means±s.e. from three biological replicates. FIG. 2E is a graph of nodule counts on roots of the miR4416-5p stable transgenic plants and the WT. FIG. 2F is a photographic illustration of the phenotypic changes in the nodule number of the miR4416-5p stable transgenic plants as compared with WT. FIG. 2G is a graph of expression levels of Le3 in the Le3 mutants and Le3-overexpression stable transgenic plants as determined by qRT-PCR. WT value was set as “1” and the others adjusted accordingly and are shown as means±s.e. from three biological replicates. FIG. 2H is a graph of nodule counts on roots of the Le3 mutants and Le3-overexpression stable transgenic plants and the WT. FIG. 2I shows photographic illustrations of the phenotypic changes in the nodule number of the Le3 mutants and Le3-overexpression in stable transgenic plants as compared with the WT. Asterisks indicate the significance level of P≤0.01 (Student's t test).

FIGS. 3A and 3B relate to miR4416-5p and that miR4416-5p in the roots is mainly transported from the shoots of a plant to regulate nodulation. FIG. 3A shows photographic illustration of the phenotypic changes in the nodule number of grafted WT, miR4416-5p STTM (STTM), and chimeric plants. FIG. 3B is a graph of nodule counts on roots of grafted WT, ST™, and chimeric plants (brackets mark rootstocks; n=36; error bars show SEM of three biological replicates; asterisks indicate the significance level of P≤0.01 (Student's t test)).

FIG. 4A is a photographic illustration of the phenotypic changes in the above-ground plant architecture of the miR4416-5p STTM stable transgenic plants as compared with WT.

FIGS. 4B-4I are graphs of plant height (FIG. 4B), primary branch number per plant (FIG. 4C), main stem node number per plant (FIG. 4D), pod number per plant (FIG. 4E), seed number per plant (FIG. 4F), 100-seed weight per plant (FIG. 4G), seed protein (FIG. 4H), and oil content (FIG. 4I) of the miR4416-5p STTM (STTM) stable transgenic plants as compared with WT (n=36; error bars show SEM of three biological replicates; comparisons used Student's t test (STTM versus WT; *P≤0.05, **P≤0.01)).

FIG. 5 depicts results showing expression levels of MIR4416b in different soybean tissues as determined by qRT-PCR. PCR products were loaded on 2% agarose gel containing SYBR™ Safe DNA Gel Stain and visualized under blue light. The genomic DNA was set as positive control.

FIG. 6A depicts a structure of the artificial MIRNA gene producing artificial miRNA identical to the wild-type miR4416-5p sequence (SEQ ID NO: 8). The artificial miRNA was modified from soybean MIR172a by replacing miR172a/miR172a* with amiR4416-5p/amiR4416-5p* that are identical/complementary to miR4416-5p.

FIG. 6B shows the nucleic acid sequence of an artificial miRNA (SEQ ID NO: 1) which produces an active miRNA illustrated in FIG. 6A. The portion of SEQ ID NO: 1 identified with (1) is the amiR4416-5p*portion, and the area identified with (2) is the amiR4416-5p portion.

FIG. 7A is a diagrammatic illustration of a miR4416-5p STTM structure. The humps along the line represent the bulge sequence within STTM sequence (SEQ ID NO: 3) that do not have complementary nucleotides within miR4416-5p sequence (see SEQ ID NO: 13 showing the mismatch between wild-type and SEQ ID NO: 3 at CTA of SEQ ID NO: 3).

FIG. 7B is the nucleic acid sequence of a miR4416-5p STTM (SEQ ID NO: 2) construct as illustrated in FIG. 7A, wherein the portion identified as (3) is a first miR4416-5p STTM sequence (SEQ ID NO: 3), the portion identified as (4) is a second miR4416-5p STTM sequence (SEQ ID NO: 3), and the area between is a spacer (SEQ ID NO: 12).

FIG. 7C is the nucleic acid sequence of a miR4416-5p STTM with the “bulge sequence” insert shown in lower case text (SEQ ID NO: 3).

FIG. 7D is the nucleic acid sequence of a miR4416-5p with a deletion (indicated as a *) in the middle as compared to WT (SEQ ID NO: 4).

FIG. 8 is a schematic depicting the mutation sites of the Le3 coding region and its amino acid changes (aa=amino acid) (SEQ ID NOS: 16, 18, and 20 showing nucleic acid sequences of the Le3 coding regions and SEQ ID NOS: 15, 17, and 19 showing sequences of the respective amino acids).

FIG. 9A is a diagrammatic illustration of miR4416-3p STTM structure. The humps along the line represent the bulge sequence within STTM sequence (SEQ ID NO: 6) that do not have complementary nucleotides within miR4416-3p WT sequence (see SEQ ID NO: 14 showing the mismatch between wild-type and SEQ ID NO: 6 at CTA of SEQ ID NO: 6).

FIG. 9B is the nucleic acid sequence of a miR4416-3p STTM (SEQ ID NO: 5) construct as illustrated in FIG. 9A, wherein the portion identified as (5) is a first miR4416-3p STTM sequence (SEQ ID NO: 6), the portion identified as (4) is a second miR4416-3p STTM sequence (SEQ ID NO: 6), and the area therebetween is a spacer (SEQ ID NO: 12).

FIG. 9C is the nucleic acid sequence of a miR4416-3p STTM with the “bulge sequence” insert shown in lower case text (SEQ ID NO: 6).

FIG. 9D is the nucleic acid sequence of a miR4416-3p with a deletion (indicated as a *) in the middle as compared to WT (SEQ ID NO: 7).

FIG. 10 is a diagrammatic illustration of eGFP-overexpression structure.

FIG. 11 is a graph of statistics of the nodule number of the miR4416-3p STTM (miR4416-3p ST™) and the corresponding empty vector induced hairy roots (eGFPOE).

FIG. 12 is Table 3 and shows the expression levels (CPM) of the miR4416b and its predicted target gene Glyma.02G156800 in uninoculated roots, 10 dpi, and 20 dpi nodules.

FIG. 13 is Table 4 and lists the primers used in the studies described below in the Examples.

FIGS. 14A and 14B relate to enhancement of yield and seed composition studies in T3-generation stable STTM lines. FIG. 14A are graphs showing yield data for three independent STTM lines in the Williams 82 genetic background (STTM) as compared with WT Williams 82 (Control). FIG. 14B is a table displaying a summary of results from a study in the yield and changes of seed compositions in the three STTM lines as compared with WT Williams 82, as measured using near infrared (NIR).

FIG. 15 is a schematic example of a construct pGES401 backbone and guide RNA (gRNA) sequences used to edit the miR4416-5p and miR4416-3p precursor gene MIR4416 (SEQ ID NO: 57). Nucleotides within frames as shown in the precursor are two gRNAs that were used for gene editing, and the dashed lines (shown in the corresponding sequences for each of the 7 mutants; SEQ ID NOS: 58-64) represent deleted sequences created by CRISPR editing.

FIG. 16 are photographs of a representative stable mutant (Mutant 1) of MIR14416 created by gene editing as described herein as compared to a WT Williams 82 control plant.

### DETAILED DESCRIPTION

For the purpose of promoting an understanding of the principles of the present disclosure, references will now be made to the embodiments illustrated in the drawings, and specific language will be used to describe the same. It will nevertheless be understood that no limitation of the scope of this disclosure is thereby intended.

Transgenic plants/cells, isolated nucleic acid constructs, and methods of enhancing plant yield are provided. The term “plant” includes whole plants, plant organs (e.g., leaves, stems, flowers, roots, reproductive organs, embryos and parts thereof, etc.), seedlings, seeds and plant cells and progeny thereof. While the class of plants of predominant focus in the present disclosure relates to legumes, it will be understood that the inventive techniques and concepts hereof is not limited to any particular class of higher plants and the methods hereof are generally as broad as the class of higher plants amenable to transformation techniques and/or that exhibit nodulation or a similar symbiotic, cross-kingdom mechanism. In certain embodiments, the plant is a leguminous plant. A “leguminous plant” as referred to herein is any member of the Fabaceae (or Leguminosae) family that can form nodules when infected with a rhizobial microorganism. In certain embodiments, the plant is a soybean cultivar.

The terms “transgenic plants” or “transgenic plant roots” or “transgenic plant cells” refers to plants that have DNA sequences not normally transcribed into RNA or translated into a protein (“expressed”), including, but not limited to genes that are perhaps not normally present, or any other genes or DNA sequences that one desires to introduce into the non-transformed plant, but which one desires to either genetically engineer or to have altered expression. It is contemplated that in some instances the genome of transgenic plants hereof will have been augmented through the stable introduction of the transgene; however, in other instances, the introduced gene will replace an endogenous sequence. A transgenic plant includes a plant/root/cell regenerated from an originally-transformed plant or cell of the present disclosure and progeny transgenic plants from later generations or crosses of a transformed plant described herein.

In certain embodiments, transgenic plants/cells, namely of legumes, and systems that exhibit increased nodule formation as compared to wild-type are provided. Methods for enhancing plant growth, seed yield, and/or seed protein concentration are also provided, which, in at least one embodiment, leverage the nucleic acid constructs hereof to advantageously affect crop yields, plant growth, and protein production.

To increase a plant's yield potential, it is important to increase the efficiency of nodulation and nitrogen fixation. Many leguminous species can utilize atmospheric dinitrogen (N₂) gas to meet their needs for nitrogen through a symbiotic relationship with nitrogen-fixing rhizobial bacteria. Specifically, N₂ from the atmosphere is converted into ammonia (NH₃), which is then assimilated into amino acids, nucleotides, and other cellular constituents such as vitamins, flavones, and hormones. Their ability to fix gaseous nitrogen makes legumes an ideal agricultural organism as their requirement for nitrogen fertilizer is reduced.

The establishment of rhizobia-legume symbiosis is dependent on recognition of signal molecules between the partners. Upon perception of plant flavonoids, rhizobia synthesize and secrete lipochitin oligosaccharides, so called Nod factors (NF), which are perceived by root Nod factor receptors (NFR) to initiate rhizobial infection and formation of nodules where symbiotic nitrogen fixation (SNF) takes place. More specifically, the NFs initiate root hair curling, which begins with the very tip of the root hair curling around the bacteria, followed by the development of infection treads that provide a pathway for the bacteria to penetrate into the cortical cells of the roots to form nodules. Nodules are relatively distinct organs among plant species, essentially representing a controlled microbial invasion of the root. Similar to the human gut, the plant provides an environment in which specific microbes can thrive. The genetic control of nodulation development is complex, with the data presented herein showing a dependency on small RNAs (sRNAs) trafficked from shoot to root.

Because SNF is resource intensive, legumes have evolved a number of mechanisms, such as autoregulation of nodulation (AON) and nitrogen regulation of nodulation, to control the number of nodules formed. AON is a negative-feedback mechanism used by legumes to restrict root nodule numbers so as to balance symbiosis and plant growth. Disruption of AON in legumes usually leads to supernodulation, which can reduce plant productivity. In both Lotus japonicus and soybean, AON involves a mobile microRNA (miRNA), which can enhance nodulation through posttranscriptional regulation of the symbiosis suppressor gene, TOO MUCH LOVE (TML), that encodes a Kelch repeat-containing F-box protein in roots. Tsikou et al. (2018), supra; Zhang et al. (2021), supra.

miRNAs have been generally described in U.S. Pat. Nos. 7,709,616 and 8,906,870, and are generally small, non-coding RNA molecules that can play crucial roles in regulating gene expression by binding to complementary sequences on target messenger RNAs (mRNAs), which usually results in gene silencing through translational repression or mRNA degradation. “Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base pairing rules. For example, for the sequence “A G T,” is complementary to the sequence “T C A.” Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion. The terms “silence,” “elimination,” and “deletion” are used interchangeably herein and mean any addition or loss of a target gene sequence of a cell genome so that the protein expression mediated by the target gene is completely removed.

Typically, miRNAs regulate gene expression post-transcriptionally. A positive-feedback mechanism was discovered between symbiosis and plant growth, mediated by the shoot-to-root trafficking of miRNA (e.g., soybean-specific miR4416-5p and miR4416-3p) in soybean cultivars. For example, it has been determined that the presence of miRNA can downregulate the expression of the Le3 (e.g., Glyma.02G156800, publicly available in the Soybase database made available online and developed by the USDA-ARS SoyBase and Legume Clade Database group at the Iowa State University), which is a lectin gene primarily expressed in developing vegetative tissues. This, taken with the miRNA promoting supernodulation, can lead to reduced plant productivity.

The present inventors discovered that silencing or downregulating certain miRNAs, such as wild-type miR4416-5p and/or miR4416-3p can lead to the upregulation of the target gene Le3 and, ultimately, enhanced plant productivity (as compared to wild-type). Further, it was unexpectedly determined that expression (e.g., overexpression) of the Le3 gene can enhance plant productivity in the shoot, while at the same time promoting symbiosis in the root to further enhance plant productivity. Indeed, the upregulation or overexpression of the identified target host gene Le3 was determined to increase plant yield, increase seed production, and increase the protein content of the seeds themselves.

In certain embodiments, silencing miR4416-5p (which was found to downregulate Le3 expression) and/or overexpressing Le3 produced soybean plants with more branches, nodes, and seeds under conventional field conditions, resulting in an up to a 47% increase in seed yield per plant, along with improved seed protein content. In certain embodiments, modulating miRNAs and expression of a target lectin gene in soybean together can elucidate a miR4416-5p-Le3 partner-mediated systemic regulation of nodulation and plant growth, create soybean lines with reduced miR4416-5p or miR4416-3p abundance, and increase Le3 expression.

The terms “overexpression” (when used in connection with a gene), and “upregulation” have the meaning ascribed thereto by one of ordinary skill in the relevant arts, which includes (without limitation) the overexpression or misexpression of a wild-type gene product that can cause mutant phenotypes and/or lead to abundant target protein expression. “Down-regulation” or “down-regulated” may be used interchangeably and refer to a decrease in the level of a marker, such as a gene, nucleic acid, metabolite, transcript, protein, or polypeptide, as compared to an established level (e.g., that of a corresponding wild-type gene).

The present studies demonstrate significant positive effects of such strategic modifications on enhancing soybean nodulation-increases in SNF and plant productivity. Accordingly, modulating (i.e., upregulating) the Le3 gene in a plant (e.g., a soybean cultivar) and/or downregulating or otherwise silencing miR4416-5p or miR4416-3p can result in, at a minimum, increased seed yield and seed protein content of a plant.

The application of the technologies, constructs, transgenic plants, and methods hereof can be leveraged to improve at least the seed yield and the protein content in a plant, such as any soybean cultivar. It is contemplated that the Le3 target gene and its activity can be regulated, for example, by using short tandem target mimics (STTMs) or other sequence disruptions, CRISPR-Cas9 gene editing, or otherwise, and used to generate stably transgenic plants capable of enhanced seed yield and protein content (as compared to a comparable wild-type plant). As used herein, “CRISPR-Cas9” means the system composed of sgRNA (guide RNA) complementarily binding to the target genome and Cas9 protein that can cut the genome gene by binding to the sgRNA and the target genome simultaneously. As a result, when the sgRNA vector and Cas9 vector are expressed temporarily in cells together concurrently, sgRNA and Cas9 protein are produced to change targeted gene sequence, leading to modification of the targeted gene at the Cas9 binding site and possible disruption of the function of the gene. Exemplified embodiments herein are for soybean cultivars, but other plants suitable for such gene regulation/CRISPR-Cas9 editing are contemplated provided the target gene is present therein.

### Transgenic Plants

These new discoveries allow for the transgenic modification of a plant to leverage the newly discovered signaling mechanisms and confer a transgenic genotype and/or phenotype to promote and increase nodule formation, plant yield, seed yield, and protein content in the seeds (as compared to wild-type). While the present disclosure focuses on leguminous plants—and, in particular soybean cultivars—as an experimental system, it will be appreciated that the inventive concepts hereof are not so limited and any species can be employed to the extent it can express a lectin gene such as, for example, Le3 and/or is modulated by miRNA.

In certain embodiments, transgenic plants (e.g., plants, roots, and/or plant cells) are provided that are engineered to upregulate or overexpress the lectin gene Le3. In certain embodiments, transgenic plants (e.g., plants, roots, and/or plant cells) are provided that are engineered to downregulate expression of miR4416-5p or miRR4416-3p.

In certain embodiments, the transgenic plant is a leguminous plant comprising at least one mutation comprising: a first mutation that upregulates expression of a Le3 gene in the leguminous plant as compared to expression in a corresponding wild-type leguminous plant; and/or a second mutation that downregulates or silences expression of microRNA (miRNA) 4416-5p or miR4416-3p. The at least one mutation can comprise both the first mutation and the second mutation.

The plant can be a soybean cultivar, for example. The plant can be Glycine max. In certain embodiments, the plant is a plant cell or plant organ. At least one mutation can result in interference with an miRNA-Le3 interaction in the plant. In certain embodiments, at least one mutation is encoded by SEQ ID NO: 1 or a functional variant thereof.

As used herein, a “functional” biological molecule is a biological molecule in a form in which it exhibits a property or activity by which it is characterized. A functional enzyme, for example, is one which exhibits the characteristic catalytic activity by which the enzyme is characterized.

The term “functional variant” refers to a sequence having substantial or significant sequence identity or similarity to the reference sequence, which functional variant retains the biological activity of the reference sequence of which it is a variant. Functional variants encompass, for example, those variants of a nucleotide or amino acid sequence (the parent sequence) that retain the ability to exhibit the properties (such as, for example, encode functional DNA, proteins, or peptides) and to a similar extent, the same extent, or to a higher extent, as the parent sequence. In reference to a nucleic acid sequence, in some embodiments a functional variant of a nucleic acid sequence is about 10% identical, about 25% identical, about 30% identical, about 50% identical, about 65% identical, about 75% identical, about 80% identical, about 90% identical, about 95% identical, or about 99% identical to the parent nucleic acid sequence.

Upregulation can be achieved or facilitated by incorporating of one or more exogenous Le3 genes into a genome of the plant, for example, using known genome editing techniques and/or gene regulatory elements. The one or more exogenous Le3 genes can integrated at a single genomic locus. The one or more exogenous Le3 genes can comprise at least two exogenous Le3 genes that are integrated at multiple genomic loci throughout the plant genome. In certain embodiments, the Le3 gene is operably linked to a promoter that upregulates expression of the Le3 gene.

The second mutation can be encoded by SEQ ID NO: 3 or SEQ ID NO: 4, or a functional variant of SEQ ID NOS: 3 or 4 (with the proviso that the functional variant is not a wild-type sequence). The second mutation can be encoded by one or more modified miR4416-5p sequences or one or more modified miR4416-3p sequences. In certain embodiments, the modified miR4416-5p sequences can each comprise a miR4416-5p short tandem target mimic (STTM) sequence and the modified miR4416-3p sequences each comprise a miR4416-3p STTM sequence. The one or more modified miR4416-5p sequence can comprise a sequence of wild-type miR4416-5p comprising an insertion or deletion therein. The one or more modified miR4416-3p sequences can comprise a sequence of wild-type miR4416-3p comprising an insertion or deletion therein. With respect to an miR4416-5p or miR4416-3p sequence comprising an insertion or deletion therein, such insertion or deletion ensures that the modified sequences comprise nucleotides that are not complementary with the wild-type miR4416-5p or miR4416-3p sequences, respectively. This can result, in certain embodiments, in a hairpin loop structure.

The modified miR4416-5p sequence can comprise a wild-type miR4416-5p sequence with a bulge sequence inserted therein. The “bulge sequence” can be any appropriate nucleic acid, and can be from about 1 to about 9 nucleic acids long (such as 1-9 nucleic acids, about 1 to 9 nucleic acids, or 1 to about 9 nucleic acids long). In certain embodiments, the bulge sequence is about 2 to about 8 nucleic acids long (such as 2-8 nucleic acids, about 2 to 8 nucleic acids, or 2 to about 8 nucleic acids long). In certain embodiments, the bulge sequence is about 3 to about 7 nucleic acids long (such as 3-7 nucleic acids, about 3 to 7 nucleic acids, or 3 to about 7 nucleic acids long). In certain embodiments, the bulge sequence is about 4 to about 6 nucleic acids long (such as 4-6 nucleic acids, about 4 to 6 nucleic acids, or 4 to about 6 nucleic acids long). The ranges specified in this paragraph are inclusive of the stated endpoints and all 1 nucleic acid increments encompassed thereby. The modified miR4416-3p sequence can comprise a wild-type miR4416-3p sequence with a bulge sequence inserted therein. In certain embodiments, the bulge sequence is CTA. In certain embodiments, the second mutation is encoded by at least two modified miR4416-5p sequences or at least two modified miR4416-3p sequences.

The second mutation can further comprise a spacer. For example, where the second mutation comprises two or more modified miR4416-5p or miR4416-3p sequences, such modified sequences can be separated by a spacer. The spacer can be any suitable spacer (e.g., comprising nucleic acids).

The spacer can comprise a length sufficient to allow for binding between two of the modified miR4416-5p sequences or two of the modified miR4416-3p sequences in a loop structure. In certain embodiments, the spacer comprises between about 3 to about 99 nucleic acids, about 5 to about 95 nucleic acids, about 10 to about 90 nucleic acids, about 15 to about 85 nucleic acids, about 20 to about 80 nucleic acids, about 25 to about 75 nucleic acids, about 30 to about 70 nucleic acids, about 35 to about 70 nucleic acids, about 40 to about 65 nucleic acids, about 45 to about 65 nucleic acids, about 50 to about 60 nucleic acids, or about 55 nucleic acids. In certain embodiments, the spacer is 40, 41, 42, 43, 44, 45, 46, 47, 48, 49, or 50 nucleic acids in length. In certain embodiments, the spacer has 48 nucleic acids. All ranges stated in this paragraph are inclusive of the stated end points and all 1 nucleic acid increments encompassed thereby.

### Expression Constructs

Isolated expression constructs are also provided. The term “isolated” means that the material is removed from its original environment, e.g., the natural environment if it is naturally occurring. For example, a naturally occurring polypeptide present within a living organism is not isolated, but the same polypeptide separated from some or all of the coexisting materials in the natural system is isolated. “Nucleic acid” refers to deoxyribonucleotides or ribonucleotides and polymers thereof in either single- or double-stranded form and complements thereof. The term encompasses nucleic acids containing known nucleotide analogs or modified backbone residues or linkages, that are synthetic, naturally occurring, and non-naturally occurring, have similar binding properties as the reference nucleic acid, and metabolized in a manner similar to the reference nucleotides.

The expression construct can comprise the nucleic acid sequence amiR4416-5p*, amiR4416-5p, and a spacer segment of nucleic acids between them, the spacer segment having enough base pairs to allow for binding between the amiR4416-5p* and amiR4416-5p nucleic acid sequences.

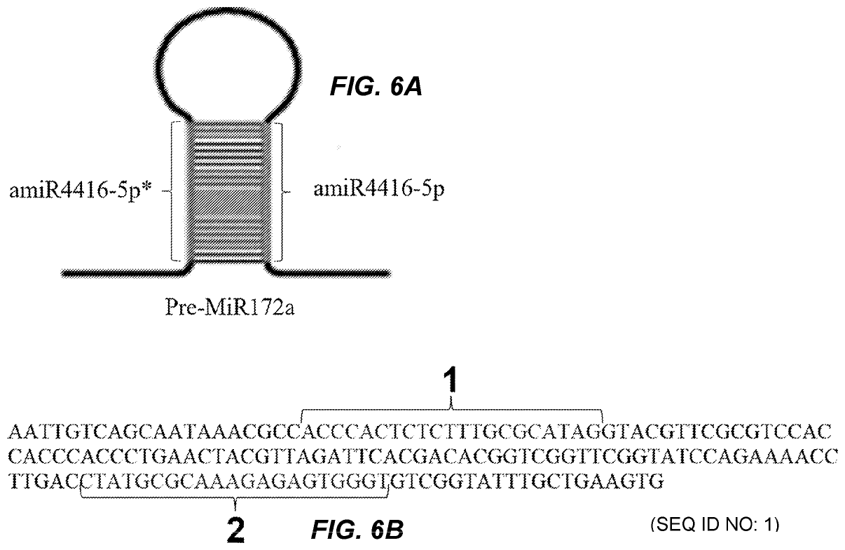

In certain embodiments, expression constructs are provided that comprise the nucleic acid sequence of amiR4416-5p* and amiR4416-5p, where the construct forms a self-binding loop structure as shown in FIG. 6A. The expression construct can comprise the nucleic acid sequence as shown in FIG. 6B (SEQ ID NO: 1) or a functional variant of SEQ ID NO: 1 (with the proviso that the functional variant is not a wild-type sequence). In certain embodiments, the expression construct has a nucleic acid sequence as shown in FIG. 6B (SEQ ID NO: 1) or a functional variant of SEQ ID NO: 1 (with the proviso that the functional variant is not a wild-type sequence). In certain embodiments, the expression construct consists of a nucleic acid sequence as shown in FIG. 6B (SEQ ID NO: 1) or a functional variant of SEQ ID NO: 1 (with the proviso that the functional variant is not a wild-type sequence).

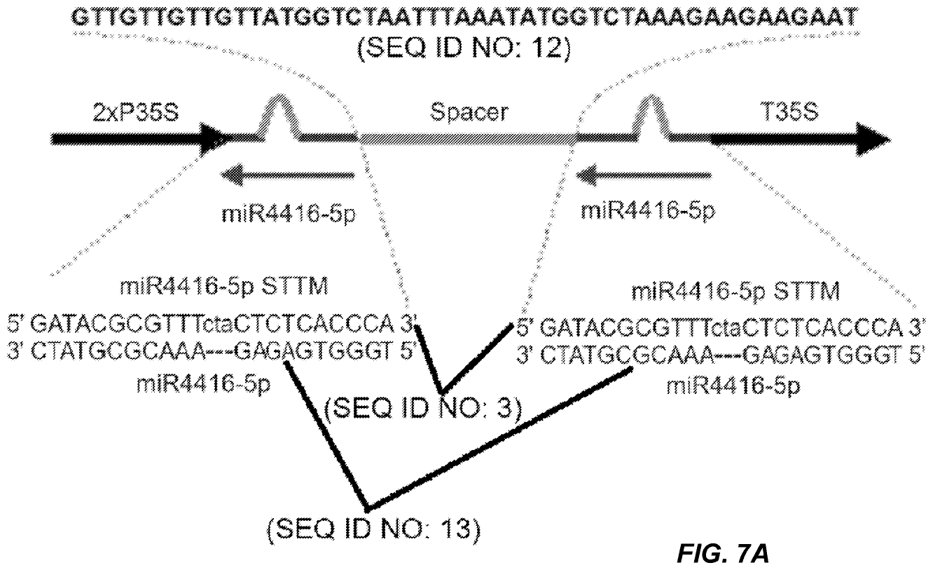

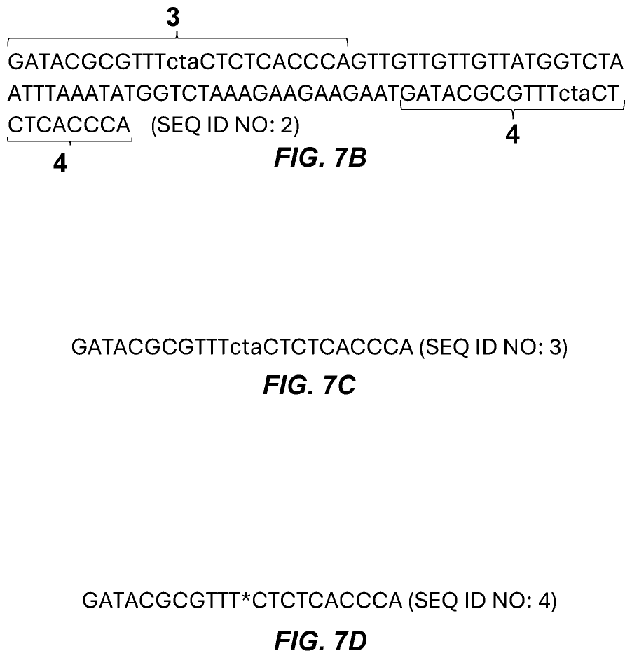

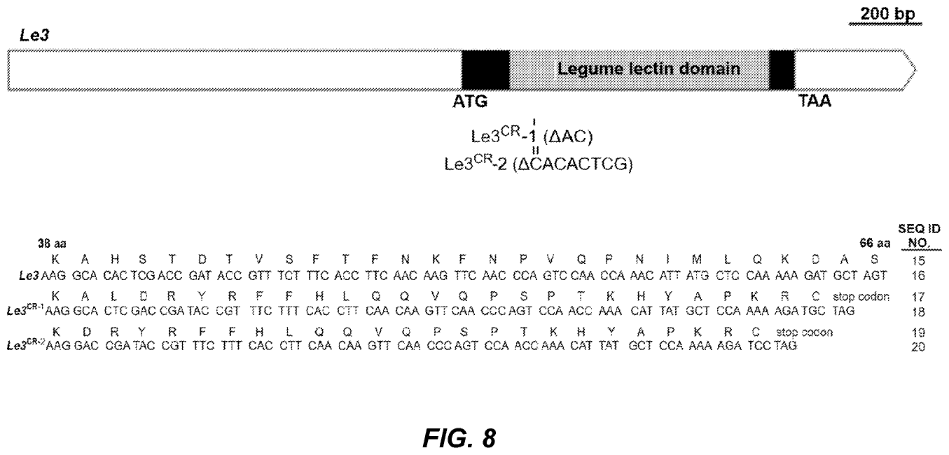

The expression construct can comprise or consist of the nucleic acid sequence of miR4416-5p STTM (SEQ ID NO: 3) as show in FIG. 7C or a functional variant of SEQ ID NO: 3 (with the proviso that the functional variant is not a wild-type miR4416-5p sequence). The expression construct can comprise or consist of the nucleic acid sequence of miR4416-5p STTM (SEQ ID NO: 4) as shown in FIG. 7D or a functional variant of SEQ ID NO: 4 (with the proviso that the functional variant is not a wild-type miR4416-5p sequence). The expression construct can comprise or consist of a nucleic acid sequence comprising a wild-type miR4416-5p or miR4416-3p sequence that has been interrupted at least once by a nucleic acid insert. In certain embodiments, the insert is or comprises CTA. The insert can be about 1 to 3 nucleic acids (such as about 1 nucleic acid to about 3 nucleic acids, 1 nucleic acid to about 3 nucleic acids, or 1-3 nucleic acids). This insert can be placed at a different location in the miR4416-5p or miR4416-3p sequence. This insert can be placed one or more times in the sequence and/or comprise different lengths in each instance.

The insert can comprise a bulge sequence. The bulge sequence can be any bulge sequence described herein. In certain embodiments, the bulge sequence of the expression construct is about 1 to about 9 nucleic acids long (such as 1-9 nucleic acids, about 1 to 9 nucleic acids, or 1 to about 9 nucleic acids long). In certain embodiments, the bulge sequence is about 2 to about 8 nucleic acids long (such as 2-8 nucleic acids, about 2 to 8 nucleic acids, or 2 to about 8 nucleic acids long). In certain embodiments, the bulge sequence is about 3 to about 7 nucleic acids long (such as 3-7 nucleic acids, about 3 to 7 nucleic acids, or 3 to about 7 nucleic acids long). In certain embodiments, the bulge sequence is about 4 to about 6 nucleic acids long (such as 4-6 nucleic acids, about 4 to 6 nucleic acids, or 4 to about 6 nucleic acids long). The ranges specified in this paragraph are inclusive of the stated endpoints and all 1 nucleic acid increments encompassed thereby.

The expression construct can comprise one or more of the nucleic acid sequences of miR4416-5p ST™, and further comprise a spacer nucleic acid sequence having a non-coding and non-hybridizing sequence of at or about 48 nucleic acids. The spacer can be any of the spacers described herein. In certain embodiments, the expression construct encodes a spacer that is about 6 to about 96 nucleic acids in length (such as about 6 to 96 nucleic acids, 6 to 96 nucleic acids, or about 6 to about 96 nucleic acids) in length. The spacer can be about 3 to about 99 nucleic acids (such as about 3 to 99 nucleic acids, 3 to 99 nucleic acids, or about 3 to about 99 nucleic acids) in length. The spacer can be about 3 to about 60 nucleic acids (such as about 3 to 60 nucleic acids, 3 to 60 nucleic acids, or about 3 to about 60 nucleic acids) in length.

In certain embodiments, the expression construct comprises a first nucleic acid sequence that upregulates expression of a Le3 gene in a leguminous plant as compared to wild-type expression in a corresponding wild-type leguminous plant; and/or a second nucleic acid sequence that downregulates or silences expression of microRNA (miRNA) 4416-5p or miR4416-3p. The expression construct can comprise SEQ ID NO: 1 or SEQ ID NO: 2, or a functional variant of SEQ ID NO: 1 or SEQ ID NO: 2 (with the proviso that the functional variant is not a wild-type sequence of miR4416-5p or miR4416-3p). In certain embodiments, the expression construct comprises both the first nucleic acid sequence and the second nucleic acid sequence. The first nucleic acid sequence of the construct can encode two or more Le3 genes. The two or more Le3 genes can be encoded at a single genomic locus or at multiple genomic loci.

In certain embodiments, the expression construct comprises or consists of the first nucleic acid sequence. The first nucleic acid sequence can comprise one or more gene regulatory elements such as, for example, one or more promoters. In certain embodiments, one or more gene regulatory elements are capable of upregulating expression of the Le3 gene as compared to wild-type expression.

In certain embodiments, the expression construct comprises or consists of the second nucleic acid sequence. The second nucleic acid sequence can comprise or be SEQ ID NO: 3 or SEQ ID NO: 4. In certain embodiments, the second nucleic acid sequence is or comprises a functional variant of SEQ ID NO: 3 and/or SEQ ID NO: 4 (with the proviso that each functional variant is not a wild-type sequence of miR4416-5p). The second nucleic acid sequence can encode one or more modified miR4416-5p sequences or one or more modified miR4416-3p sequences. The modified miR4416-5p sequences each comprise a miR4416-5p STTM sequence. The one or more modified miR4416-5p sequences can, for example, comprise a sequence of wild-type miR4416-5p with an insertion or deletion therein. In certain embodiments, the one or more modified miR4416-5p sequences can, for example, comprise a sequence of wild-type miR4416-5p with a bulge sequence inserted therein (e.g., a bulge sequence as previously described). The bulge sequence can be or comprise CTA.

The modified miR4416-3p sequences can each comprise a miR4416-3p STTM sequence. The modified miR4416-3p sequences each comprise a miR4416-3p STTM sequence. The one or more modified miR4416-3p sequences can, for example, comprise a sequence of wild-type miR4416-3p with an insertion or deletion therein. In certain embodiments, the one or more modified miR4416-3p sequences can, for example, comprise a sequence of wild-type miR4416-3p with a bulge sequence inserted therein (e.g., a bulge sequence as previously described). The bulge sequence can be CTA or comprise.

In certain embodiments, where the expression construct comprises or consists of the second nucleic acid sequence, the second nucleic acid sequence comprises two or more modified miR4416-5p sequences or two or more modified miR4416-3p sequences. In each such case, each of the modified miR4416-5p or miR4416-3p sequences can be, for example, separated by a spacer. The spacer can comprise any suitable spacer. The spacer can be any spacer described herein. The spacer can, for example, be of a length sufficient to allow for binding between two of the modified miR4416-5p sequences or two of the modified miR4416-3p sequences in a loop structure. The spacer can comprise between about 3 to about 99 nucleic acids and, optionally, about 48 nucleic acids.

In certain embodiments, the expression construct is or comprises SEQ ID NO: 1, 2 or 5 or a functional variant of SEQ ID NO: 1, 2 or 5 (with the proviso that the functional variant is not a wild-type MIRNA gene).

### Genome Editing Systems

The expression constructs can be used for genome engineering. The phrase “used for genome engineering” refers to the incorporation of targeting nucleic acid sequences, nucleic acid markers, and other such gene editing components into the expression constructs and transgenic plants hereof. Such genomic editing tools include, without limitation, CRISPR (Clustered Regularly Interspaced Short Palindromic Repeats) nucleic acid sequences. The expression constructs can be useful in an analogous way as the CRISPR-Cas9 genome editing tool for editing plant genomes.

Also provided are genome editing systems that comprise the expression constructs hereof. In certain embodiments, the genome editing system comprises any of the expression constructs described.

The genome editing system further comprises a sequence-specific nuclease, a DNA polymerase, and/or a DNA polymerase recruitment protein, or an expression construct comprising a nucleotide sequence encoding the sequence-specific nuclease, the DNA polymerase and/or the DNA polymerase recruitment protein.

Targeted CRISPR-Cas9 mechanisms are known in the art and can be incorporated into the genome editing system hereof. The sequence specific nuclease can be a CRISPR nuclease, such as a CRISPR nickase. The CRISPR nickase can be a Cas9 nickase. In certain embodiments, the expression construct comprising a nucleotide sequence encoding the sequence-specific nuclease comprises a T-DNA binary vector and the sequence specific nuclease is a Cas9 nickase.

A “vector” is a composition of matter which comprises an isolated nucleic acid sequence, which can be used to transfer gene sequences between cells. Thus, the term includes cloning and expression vehicles, as well as viral vectors. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

The DNA polymerase can comprise (or be part of) a primer-based PCR system as is known in the art.

The genome editing system can further comprise a guide RNA and/or an expression construct comprising a nucleotide sequence encoding the guide RNA. In certain embodiments, the genome editing system further comprises a guide RNA protospacer that targets miR1446-5p or miR1446-3p.

In certain embodiments of the genome editing system, the expression construct is or comprises SEQ ID NO: 1, 2 or 5 or a functional variant of SEQ ID NO: 1, 2 or 5 (with the proviso that the functional variant is not a wild-type MIRNA gene), and the gene editing system, in use, affects CRISPR mutagenesis about 50% to about 80% as well as compared to a genome editing system employing a wild-type soybean strain (such as about 50% to 80%, 50% to about 80%, or 50% to 80% as well). In certain embodiments, the genome editing system affects CRISPR mutagenesis about 55% to about 75% as well as compared to a genome editing system employing a wild-type soybean strain (such as about 55% to 75%, 55% to about 75%, or 55% to 75% as well). In certain embodiments, the genome editing system affects CRISPR mutagenesis about 60% to about 70% as well as compared to a genome editing system employing a wild-type soybean strain (such as about 60% to 70%, 60% to about 70%, or 60% to 70% as well). In certain embodiments, the genome editing system affects CRISPR mutagenesis about 55% to about 78% as well as compared to a genome editing system employing a wild-type soybean strain (such as about 55% to 78%, 55% to about 78%, or 55% to 78% as well). The ranges set forth in this paragraph are inclusive of the stated end points and include all 1% increments encompassed therein.

### Methods and Uses

A method of making and using the transgenic plants and expression constructs are also provided herein. The expression constructs can be used for gene editing in any leguminous plant species. The expression constructs can be used for gene editing in any plant species that expresses Le3. In certain embodiments, the expression constructs are used with CRISPR/Cas genome editing.

Methods enhancing plant growth are also provided, for example, using the transgenic plants (e.g., plant cells) that are engineered to upregulate, repress, downregulate, or silence the identified targets. In certain embodiments, the method for enhancing plant growth comprises upregulating expression of a Le3 gene in the plant as compared to expression in a corresponding wild-type leguminous plant; and/or downregulating or silencing expression of miR4416-5p or miR4416-3p. In certain embodiments, the method comprises both upregulating expression of a Le3 gene and downregulating or silencing expression of miR4416-5p or miR4416-3p.

The plant can be a whole plant, a plant organ, or a plant cell. The plant can be a soybean cultivar. The plant can be a leguminous plant. The plant can be Glycine max.

Upregulating expression of a Le3 gene and/or downregulating or silencing expression of miR4416-5p or miR4416-3p of the plant can comprise introducing one or more of the expression constructs hereof into the plant (e.g., a plant cell) or incorporating modifications into a sequence of miR4416-5p and/or a sequence of miR4416-3p of the plant.

Upregulating expression of a Le3 gene can comprise incorporating one or more Le3 genes into a genome of the plant. The one or more Le3 genes can be exogenous. The one or more Le3 genes can be integrated at a single genomic locus of the plant. The one or more Le3 genes can comprises at least two exogenous Le3 genes that are integrated at multiple genomic loci. The Le3 gene can be operably linked to a promotor (or one or more promotors) that upregulates expression of the Le3 gene. Upregulating can comprise incorporating one or more gene regulatory elements into a genome of the plant (e.g., such as one or more promoters).

In certain embodiments, downregulating or silencing expression of miR4416-5p or miR4416-3p comprises transforming the plant with a construct comprising SEQ ID NO: 3 or SEQ ID NO: 4, or a functional variant of SEQ ID NO: 3 or SEQ ID NO: 4 (with the proviso that the functional variant is not a wild-type miR4416-5p sequence). In certain embodiments, downregulating or silencing expression of miR4416-5p or miR4416-3p comprises transforming the plant with a construct comprising SEQ ID NO: 6 or SEQ ID NO: 7, or a functional variant of SEQ ID NO: 6 or SEQ ID NO: 7 (with the proviso that the functional variant is not a wild-type miR4416-3p sequence). In certain embodiments, downregulating or silencing expression of miR4416-5p and/or miR4416-3p comprises transforming the plant with a construct comprising SEQ ID NO: 3, SEQ ID NO: 4, SEQ ID NO: 6, SEQ ID NO: 7, or two or more of SEQ ID NOS: 3, 4, 6, or 7, or a functional variant of SEQ ID NOS: 3, 4, 6, or 7, and/or two or more functional variants of SEQ ID NOS: 3, 4, 6, or 7.

Downregulating or silencing expression of miR4416-5p or miR4416-3p can comprise transforming the plant with an expression construct comprising one or more modified miR4416-5p sequences or one or more modified miR4416-3p sequences. Optionally, the modified miR4416-5p sequences can each comprise a miR4416-5p STTM sequence and/or the modified miR4416-3p sequences each comprise a miR4416-3p STTM sequence. The one or more modified miR4416-5p sequence can comprise a sequence of wild-type miR4416-5p having an insertion or deletion therein. The modified miR4416-5p sequence can comprise a wild-type miR4416-5p sequence with a bulge sequence inserted therein. The bulge sequence can be CTA or any other suitable bulge sequence.

The one or more modified miR4416-3p sequence can comprise a sequence of wild-type miR4416-3p with an insertion or deletion therein. The modified miR4416-3p sequence can comprise a wild-type miR4416-3p sequence with a bulge sequence inserted therein. The bulge sequence can be CTA or any other suitable bulge sequence.

Introducing or incorporating can be performed by gene editing techniques such as, optionally, CRISPR-Cas9, transcription activator-like effector nucleases (TALENS), homologous recombination, or any other suitable gene editing technique now known or hereinafter developed. In certain embodiments, incorporating an expression construct into the plant comprises incorporating an insertion into a sequence of miR4416-5p or miR4416-3p of the plant. In certain embodiments, introducing one or more expression constructs into the plant further comprises transforming the leguminous plant with one or more of the expression constructs.

Incorporating can be performed prior to planting the plant or after planting the plant.

Using the methods hereof, the transgenic plant yield can be enhanced by at or more than about 20% as compared to plant yield of a corresponding wild-type plant. Additionally or alternatively, the seed protein concentration of the transgenic plant can be increased as compared to seed protein concentration in a corresponding wild-type plant.

In certain embodiments, upregulating, downregulating or silencing is performed by introducing one or more expression constructs hereof into a plant (e.g., using techniques well known in the art). The one or more expression constructs can, for example, an expression construct described herein. Introducing can be performed prior to planting the plant. Introducing can be performed after planting the plant.

In at least one embodiment, the method further comprises inoculating, or having inoculated, the modified plant with at least one rhizobium to initiate nodulation. The inoculating step can be performed pursuant to known methodologies and using conventional microorganism species and strains. In one embodiment, the roots can be wounded to enable the bacterial cells to penetrate the roots more quickly and easily; however, wounding of the roots is not required.

In certain embodiments, the plant is Glycine max.

The species or strains of the at least one rhizobium can comprise any microorganism that initiates nodulation in a host plant root or cell including, for example, Bradyrhizobium japonicum, Rhizobium etli, Sinorhizobium meliloti, Rhizobium leguminosarum, Parasponia rhizobium, Mesorhizobium loti and the like.

### General

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

In the above description, numerous specific details are set forth to provide a thorough understanding of the present disclosure. Particular examples may be implemented without some or all of these specific details and it is to be understood that, unless otherwise stated, aspects hereof are not limited to particular biological systems or particular species of bacteria or plants, which can, of course, vary but remain applicable in view of the data provided herein.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While aspects of the present disclosure can be described and claimed in a particular statutory class, such as the system statutory class, this is for convenience only and one of skill in the art will understand that each aspect of the present disclosure can be described and claimed in any statutory class.

It should be emphasized that the following disclosures are put forth so as to provide those of ordinary skill in the art with a complete disclosure and description of how the composition of matter, e.g., the mutant strains in this disclosure, and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

As used in the specification and the appended claims, the singular forms “a,” “an” and “the” include plural referents unless the context clearly dictates otherwise. Thus, for example, reference to “a short chain fatty acid,” “a carnitine derivative,” or “an adjuvant,” includes, but is not limited to, combinations of two or more such short chain fatty acids, carnitine derivatives, or adjuvants, and the like.

The terms “about,” “approximate,” “at or about,” and “substantially,” when referring to a number or a numerical value or range (including, for example, whole numbers, fractions, and percentages), means that the number or numerical range referred to is an approximation within experimental variability (or within statistical experimental error). That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±1%-15% variation of the stated number or numerical range (e.g., +/−5% to 15% of the recited value), provided that one of ordinary skill in the art would consider equivalent to the recited value (e.g., having the same function or result) and unless otherwise indicated or inferred. Similarly, when values are expressed as approximations, by use of the antecedent “about,” it will be understood that the particular value forms a further aspect. For example, if the value “about 10” is disclosed, then “10” is also disclosed.

When ratios, ranges, concentrations, amounts, and other numerical data are expressed herein in a range format, all combinations and sub-combinations of such ranges and specific embodiments therein are intended to be included. It will be further understood that the endpoints of each of the ranges are significant both in relation to the other endpoint, and independently of the other endpoint.

Additionally, it is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms. Likewise, the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

It is therefore intended that this description and the appended claims will encompass all modifications and changes apparent to those of ordinary skill in the art based on this disclosure. Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

Further, the use of headings and subheadings is for ease of reference, given the length of the document. Description under one heading or subheading (such as a subheading in the Detailed Description) is not intended to be limited to only the subject matter set forth under that particular heading or subheading.

### ENUMERATED CLAUSES

Clause 1. A leguminous plant comprising at least one mutation, the at least one mutation comprising: a first mutation that upregulates expression of a Le3 gene in the leguminous plant as compared to expression in a corresponding wild-type leguminous plant; and/or a second mutation that downregulates or silences expression of microRNA (miRNA) 4416-5p or miR4416-3p.

Clause 2. The leguminous plant of clause 1, wherein the at least one mutation comprises both the first mutation and the second mutation.

Clause 3. The leguminous plant of clause 1 or 2, wherein the plant is a soybean cultivar.

Clause 4. The leguminous plant of any one of clauses 1-3, wherein the upregulation is facilitated by the incorporation of one or more exogenous Le3 genes into a genome of the plant.

Clause 5. The leguminous plant of clause 4, wherein the one or more exogenous Le3 genes are integrated at a single genomic locus.

Clause 5. The leguminous plant of clause 4, wherein the one or more exogenous Le3 genes comprises at least two exogenous Le3 genes that are integrated at multiple genomic loci.

Clause 6. The leguminous plant of any one of clauses 1-5, wherein upregulation is facilitated by downregulating or silencing expression of miRNA 4416-5p or miR4416-3p.

Clause 8. The leguminous plant of any one of clauses 1-7, wherein the Le3 gene is operably linked to a promoter that upregulates expression of the Le3 gene.

Clause 9. The leguminous plant of any one of clauses 1-8, wherein the upregulation is facilitated by the incorporation of one or more gene regulatory elements.

Clause 10. The leguminous plant of any one of clauses 1-9, wherein the plant is a plant cell.

Clause 11. The leguminous plant of any one of clauses 1-10, wherein the second mutation is encoded by SEQ ID NO: 3 or SEQ ID NO: 4, or a functional variant of SEQ ID NO: 3 or SEQ ID NO: 4 (with the proviso that the functional variant is not a wild-type miR4416-5p sequence).

Clause 12. The leguminous plant of any one of clauses 1-10, wherein the second mutation is encoded by one or more modified miR4416-5p sequences or one or more modified miR4416-3p sequences, and, optionally wherein: the modified miR4416-5p sequences each independently comprise a miR4416-5p short tandem target mimic (STTM) sequence or a sequence of wild-type miR4416-5p comprising an insertion or deletion therein, and/or the modified miR4416-3p sequences each comprise a miR4416-3p STTM sequence or a sequence of wild-type miR4416-3p comprising an insertion or deletion therein.

Clause 13. The leguminous plant of clause 12, wherein the modified miR4416-5p sequence comprises a wild-type miR4416-5p sequence with a bulge sequence inserted therein.

Clause 14. The leguminous plant of clause 12, wherein the modified miR4416-3p sequence comprises a wild-type miR4416-3p sequence with a bulge sequence inserted therein.

Clause 15. The leguminous plant of clause 13 or 14, wherein the bulge sequence is or comprises CTA.

Clause 16. The leguminous plant of any one of clauses 1-15, wherein the second mutation is encoded by at least two modified miR4416-5p sequences and/or at least two modified miR4416-3p sequences, each of the modified miR4416-5p or miR4416-3p sequences separated by a spacer, wherein, optionally: each of the at least two modified miR4416-5p sequences independently comprises SEQ ID NO: 3 or SEQ ID NO: 4, or a functional variant of SEQ ID NO: 3 or SEQ ID NO: 4; and/or each of the at least two modified miR4416-3p sequences independently comprises SEQ ID NO: 6 or SEQ ID NO: 7, or a functional variant of SEQ ID NO: 6 or SEQ ID NO: 7.

Clause 17. The leguminous plant of clause 16, wherein the spacer is of a length sufficient to allow for binding between two of the modified miR4416-5p sequences or two of the modified miR4416-3p sequences in a loop structure.

Clause 18. The leguminous plant of clause 16, wherein the spacer comprises between about 3 to about 99 nucleic acids and is optionally about 48 nucleic acids.

Clause 19. The leguminous plant of any one of clauses 1-18, wherein the at least one mutation results in interference with a miRNA-Le3 interaction in the plant.

Clause 20. The leguminous plant of any one of clauses 1-10, 12-14, and 16-19, wherein the at least one mutation is encoded by SEQ ID NO: 1 or a functional variant of SEQ ID NO: 1.

Clause 21. An expression construct comprising: a first nucleic acid sequence that upregulates expression of a Le3 gene in a leguminous plant as compared to wild-type expression in a corresponding wild-type leguminous plant; and/or a second nucleic acid sequence that downregulates or silences expression of microRNA (miRNA) miR4416-5p or miR4416-3p.

Clause 22. The expression construct of clause 21, wherein the expression construct comprises both the first nucleic acid sequence and the second nucleic acid sequence.

Clause 23. The expression construct of clause 21, comprising SEQ ID NO: 1, 2, or 5 or a functional variant of SEQ ID NO: 1, 2, or 5.

Clause 24. The expression construct of claim 21 or 22, comprising the first nucleic acid sequence, wherein the first nucleic acid sequence encodes two or more Le3 genes.

Clause 25. The expression construct of clause 24, wherein the two or more Le3 genes are encoded at a single genomic locus.

Clause 26. The expression construct of clause 24, wherein the two or more Le3 genes are encoded at multiple genomic loci.

Clause 27. The expression construct of any one of clauses 21-26, comprising the first nucleic acid sequence, wherein the first nucleic acid sequence comprises one or more gene regulatory elements.

Clause 28. The expression construct of clause 27, wherein the one or more gene regulatory elements comprise at least a promoter for upregulating expression of the Le3 gene as compared to wild-type expression.

Clause 29. The expression construct of any one of clauses 21-28, comprising the second nucleic acid sequence, wherein the second nucleic acid sequence comprises SEQ ID NO: 3 or SEQ ID NO: 4, or a functional variant of SEQ ID NO: 3 or 4 (with the proviso that the functional variant is not a wild-type sequence of miR4416-5p).

Clause 30. The expression construct of any one of clauses 21-29, comprising the second nucleic acid sequence, wherein the second nucleic acid sequence comprises one or more modified miR4416-5p sequences or one or more modified miR4416-3p sequences, and, optionally wherein: at least one of the modified miR4416-5p sequences comprises a miR4416-5p STTM sequence or a sequence of wild-type miR4416-5p comprising an insertion or deletion therein; and at least one of the modified miR4416-3p sequences comprises a miR4416-3p STTM sequence or a sequence of wild-type miR4416-3p comprising an insertion or deletion therein.

Clause 31. The expression construct of clause 30, wherein the one or more modified miR4416-5p sequences comprises a wild-type miR4416-5p sequence with a bulge sequence inserted therein.

Clause 32. The expression construct of clause 30, wherein the one or more modified miR4416-3p sequences comprises a wild-type miR4416-3p sequence with a bulge sequence inserted therein.

Clause 33. The expression construct of clauses 31 or 32, wherein the bulge sequence is or comprises CTA.

Clause 34. The expression construct of clause 21, comprising the second nucleic acid sequence, wherein the second nucleic acid sequence encodes two or more miR4416-5p or two or more modified miR4416-3p, wherein each of the modified miR4416-5p or miR4416-3p sequences is separated by a spacer.

Clause 35. The expression construct of clause 34, wherein the spacer is of a length sufficient to allow for binding between two of the modified miR4416-5p sequences or two of the modified miR4416-3p sequences in a loop structure.

Clause 36. The expression construct of clause 34, wherein the spacer comprises between about 3 to about 99 nucleic acids and is optionally about 48 nucleic acids.

Clause 37. A method for enhancing leguminous plant growth comprising: upregulating expression of a Le3 gene in a plant as compared to expression in a corresponding wild-type leguminous plant; and/or downregulating or silencing expression of microRNA miR4416-5p, miR4416-3p, or both miR4416-5p and miR4416-3p in the plant as compared to expression in a corresponding wild-type leguminous plant.

Clause 38. The method of clause 37, wherein the method comprises both upregulating expression of a Le3 gene and downregulating or silencing expression of miR4416-5p or miR4416-3p.

Clause 39. The method of clause 37 or 38, wherein upregulating expression of a Le3 gene and/or downregulating or silencing expression of miR4416-5p and/or miR4416-3p comprises introducing one or more expression constructs of any one of claims 21-36 into the plant or incorporating modifications into a sequence of miR4416-5p and/or the sequence of miR4416-3p of the plant.

Clause 40. The method of clause 39, wherein upregulating expression of a Le3 gene comprises incorporating one or more exogenous Le3 genes into a genome of the plant.

Clause 41. The method of clause 40, wherein the one or more exogenous Le3 genes are integrated at a single genomic locus.

Clause 42. The method of clause 40, wherein the one or more exogenous Le3 genes comprises at least two exogenous Le3 genes that are integrated at multiple genomic loci.

Clause 43. The method of any one of clauses 37-42, wherein the Le3 gene is operably linked to a promoter that upregulates expression of the Le3 gene.

Clause 44. The method of any one of clauses 37-43, wherein upregulating comprises incorporating one or more gene regulatory elements into a genome of the plant.

Clause 45. The method of any one of clauses 37-44, wherein the plant is a plant cell.

Clause 46. The method of any one of clauses 37-45, wherein the downregulating or silencing expression of miR4416-5p and/or miR4416-3p comprises transforming the plant with a construct comprising SEQ ID NO: 3, SEQ ID NO: 4, SEQ ID NO: 6, SEQ ID NO: 7, or two or more of SEQ ID NOS: 3, 4, 6, or 7.

Clause 47. The method of any one of clauses 37-45, wherein downregulating or silencing expression of miR4416-5p and/or miR4416-3p comprises transforming the plant with an expression construct comprising one or more modified miR4416-5p sequences or one or more modified miR4416-3p sequences, and, optionally wherein: at least one of the modified miR4416-5p sequences comprises a miR4416-5p STTM sequence or a sequence of wild-type miR4416-5p comprising an insertion or deletion therein; and at least one of the modified miR4416-3p sequences comprises a miR4416-3p STTM sequence or a sequence of wild-type miR4416-3p comprising an insertion or deletion therein.

Clause 48. The method of clause 47, wherein the one or more modified miR4416-5p sequence comprises a wild-type miR4416-5p sequence with a bulge sequence inserted therein.

Clause 49. The method of clause 47, wherein the one or more modified miR4416-3p sequence comprises a wild-type miR4416-3p sequence with a bulge sequence inserted therein.

Clause 50. The method of clause 48 or 49, wherein the bulge sequence is or comprises CTA.

Clause 51. The method of clause 39, wherein introducing or incorporating is performed by gene editing techniques such as, optionally, CRISPR-Cas9, TALENs, or homologous recombination.

Clause 52. The method of clause 51, wherein incorporating comprises incorporating an insertion into a sequence of miR4416-5p or miR4416-3p of the plant.

Clause 53. The method of clause 39, wherein introducing one or more expression constructs into the plant further comprises transforming the leguminous plant with one or more of the expression constructs.

Clause 54. The method of any one of clauses 37-53, wherein plant yield is enhanced by at or more than about 20% as compared to plant yield of a corresponding wild-type plant.

Clause 55. The method of any one of clauses 37-54, wherein seed protein concentration is increased as compared to seed protein concentration in a corresponding wild-type plant.

Clause 56. The method of clause 39, wherein introducing or incorporating is performed prior to planting the plant or after planting the plant.

Clause 57. The method of any one of clauses 37-56, further comprising inoculating, or having inoculated, the plant with at least one rhizobium to initiate nodulation.

### EXAMPLES

The following examples serve to illustrate aspects of the present disclosure. The examples are not intended to limit the scope of the claimed invention in any way.

For the studies described below, unless otherwise specified, soybean (Glycine max) cultivar Williams 82, stable transgenic soybean plants and the composite soybean plants with hairy root were grown in the greenhouse under 16 hours light/8 hours dark cycles at 25° C. (day)/20° C. (night). Bradyrhizobium diazoefficiens USDA 110 was cultured in a modified arabinose gluconate (MAG) medium P. van Berkum et al. 1990) and used for soybean (hairy) root inoculation.

Genomic DNA isolation, polymerase chain reaction (PCR), quantitative real-time PCR (qRT-PCR), sequencing of DNA fragments and PCR products were performed as previously described in Zhang et al., Elevation of soybean seed oil content through selection for seed coat shininess, Nature Plants, 4:30-35 (2018). The soybean gene GmELF1b (Glyma.02G276600) was used as an internal reference to quantify the relative expression levels of the soybean transcripts from three biological replicates.

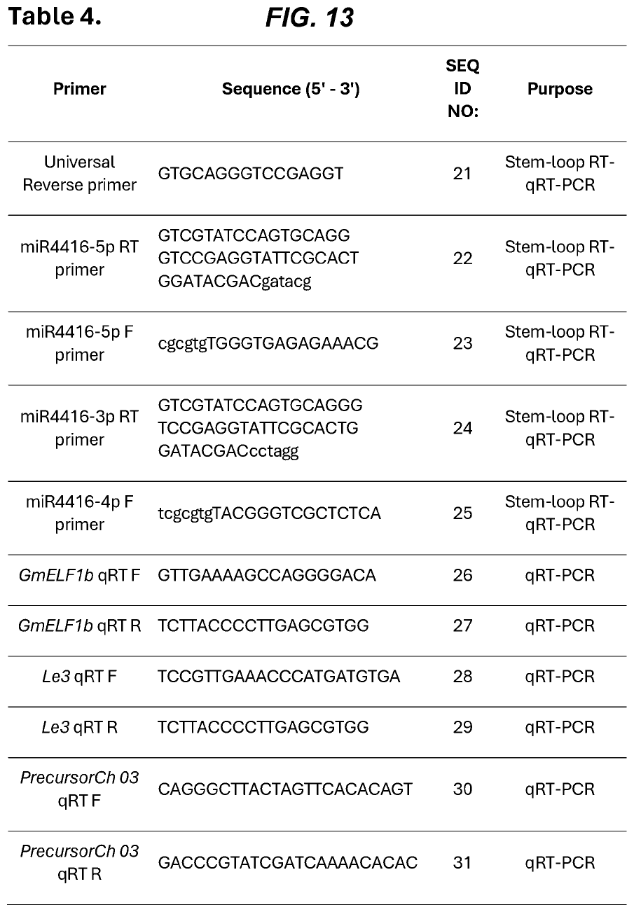

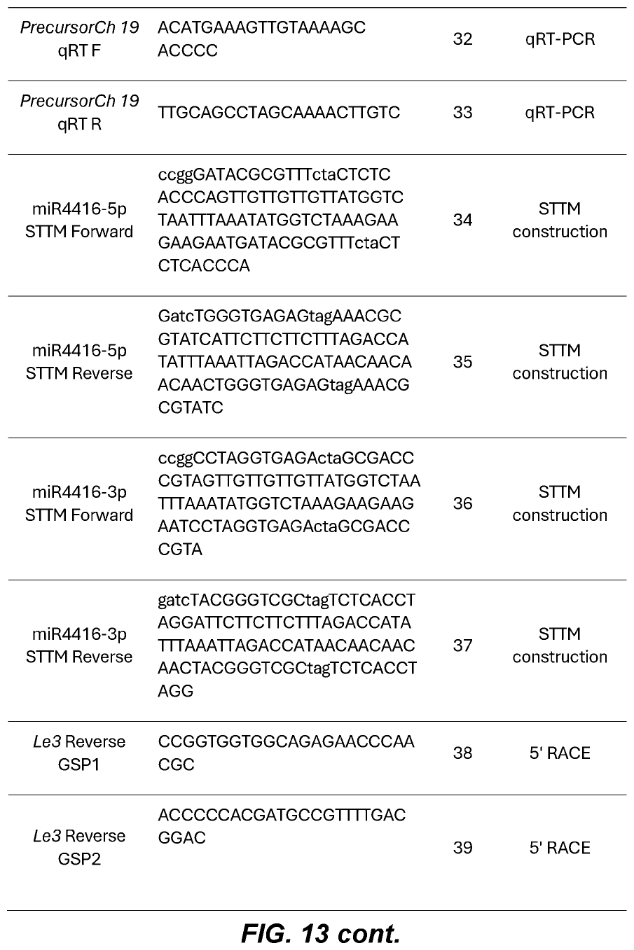

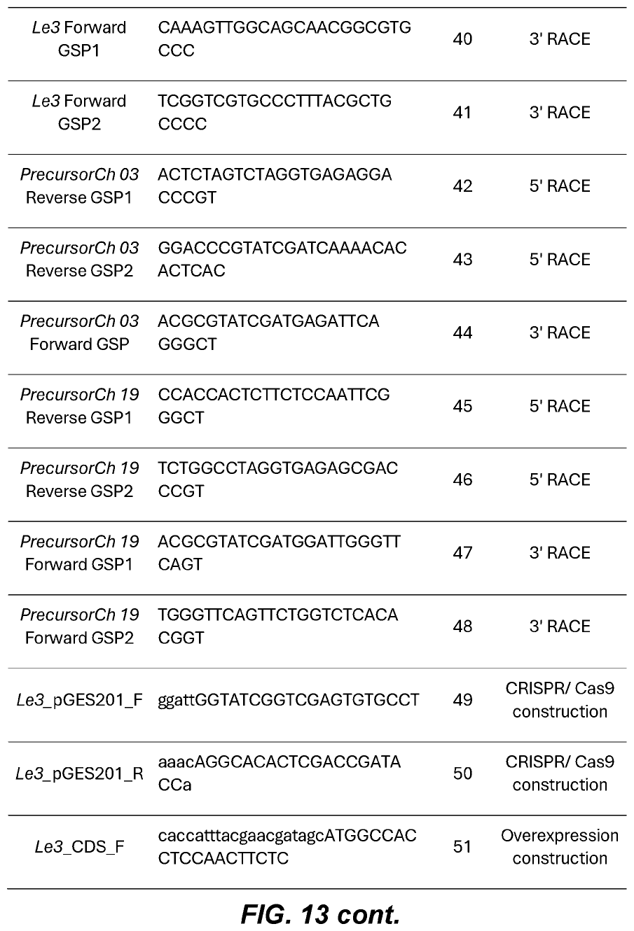

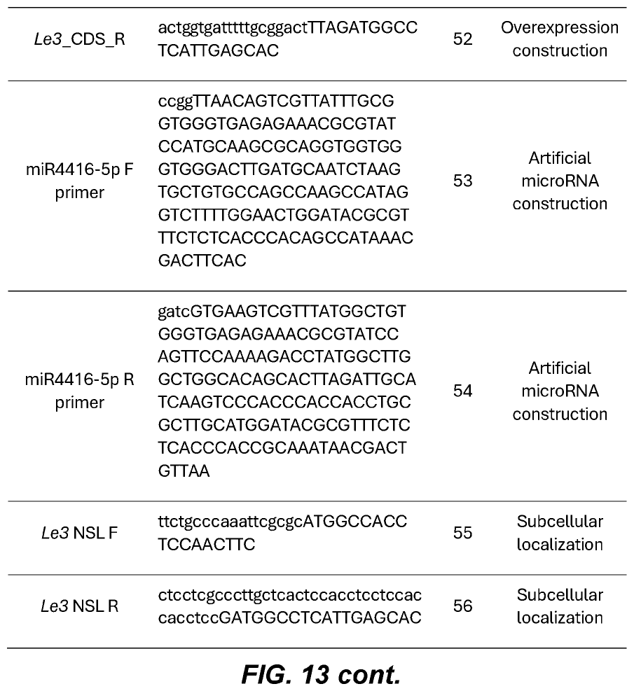

Primers used for the variety of PCR studies described are listed in Table 4 of FIG. 13.

### Example 1

### Downregulation of Le3 by Mobile miRNA

Using a shoot-root grafting system, dozens of shoot-to-root trafficking microRNAs (miRNAs) were identified in soybean (previously characterized using soybean shoot-to-root mobile small RNAs (sRNAs). Li et al., Unidirectional movement of small RNAs from shoots to roots in interspecific heterografts, Nature Plants 7:50-59 (2021). The sRNA-seq and mRNA-seq data were from soybean uninoculated root, 10-day post inoculation (dpi) nodule, and 20 dpi nodule as described in Ren et al. (2019), supra, and as made publicly available through the National Center for Biotechnology Information (NCBI, PRJNA495327).

Among these, a soybean specific 21-nt miRNA miR4416b was an attractive candidate for studies due to its relative abundance, as assessed by small RNA-sequencing, was greatly reduced in developing nodules as compared with that in roots, whereas the expression levels of the predicted miR4416b target gene Glyma.02G156800, as measured by RNA-sequencing, was substantially increased in the former as compared with that in the latter (FIG. 12). More specifically, miR4416b was selected based on the following standards: 1) being identified as shoot-to-root mobile miRNAs in soybean; 2) displaying at least a 3-fold decrease of abundance (CPM) in 10 dpi and 20 dpi nodules as compared to those in uninoculated roots.

According to miRBase, miR4416b was annotated to be produced from a putative miRNA precursor MIR4416b located on chromosome 3 of the Williams 82 soybean reference genome. Kozomara et al., miRbase: From microRNA sequences to function, Nucleic Acids Research 47: d155-d162 (2019); Schmutz et al., Genome sequence of the palaeopolyploid soybean, Nature 463:178-183 (2010). To verify this, miR4416b's precursor (Chromosome 03) was cloned by RACE and the secondary structure of miRNA precursor MIR4416 was predicted using the RNAfold server incorporated in the ViennaRNA Web Services (publicly available through the ViennaRNA Web Services website, supported by the Institute for Theoretical Chemistry).

Normal or nested 5′ and 3′ RACE products were obtained using the GeneRacer™ Kit (Invitrogen Corporation, Waltham, MA) following the manufacture's protocol. Briefly, DNase-treated total RNA from the uninoculated roots of Williams 82 was treated with calf intestinal phosphatase (CIP) to remove all active 5′ monophosphates from truncated or otherwise degraded mRNA as well as non-mRNA, rendering those RNAs unavailable for ligation and leaving only intact capped mRNA unaffected. Subsequently, tobacco acid pyrophosphatase (TAP) was used to remove the 5′ cap structure leaving a unique active 5′ phosphate on mature mRNA by hydrolyzing the pyrophosphate bonds on the m7G cap triphosphate bridge. GeneRacer™ RNA Oligo was ligated to the unique active 5′ phosphate using a T4 RNA ligase in a 10 μL reaction containing dephosphorylated, decapped RNA and 0.25 μg GeneRacer™ RNA Oligo, 1×Ligase Buffer, 1 mM ATP, 40 U RNaseOut and 5 U T4 RNA ligase.

First-strand cDNA was obtained by reverse transcription of the ligated mRNA using the GeneRacer™ Oligo dT Primer. For 5′ RACE, two rounds of PCR amplification were performed with forward primers located within the GeneRacer™ RNA Oligo (GeneRacer™ 5′ Primer and GeneRacer™ 5′ Nested Primer) and reverse primers in each transcript (Table 4, FIG. 13). For 3′ RACE, one/two round(s) of PCR amplification was/were performed with forward primer(s) located within each transcript (Table 4, FIG. 13) and reverse primers in GeneRacer™ Oligo dT (GeneRacer™ 3′ Primer and GeneRacer™ 3′ Nested Primer). Nested PCRs were performed using a 1:50 dilution of the first round PCR product as a template. The PCR products were cloned into the pCR™4Blunt-TOPO™ vector (Invitrogen Corporation, Waltham, MA) and sequenced at Eurofins Genomics (Louisville, KY) to experimentally confirm the transcription start sites (TSSs) and transcription termination sites (TTSs) that define the full-length transcripts. To clone miR4416b's precursor (Chromosome 03) by RACE, primers were first designed according to the MIR4416b stem-loop sequence listed in miRbase.

Surprisingly, when measured by qRT-PCR, the expression of this putative precursor was not detected in any of the tissues collected from Williams 82 with the first trifoliolate (i.e., at the V1 stage), including developing roots, cotyledons, leaves and shoot apexes, as examined by quantitative reverse-transcription PCR (qRT-PCR) (FIG. 5). More specifically, no product was acquired after PCR and the qRT-PCR failed to produce any specific product when referring to the same stem-loop sequence in different soybean tissues. This was consistent with the RACE results, which supported very low to no expression of the miR4416b's precursor (Chromosome 03). Therefore, the annotated “MIR4416b” was deemed unlikely to be the precursor of miR4416b.

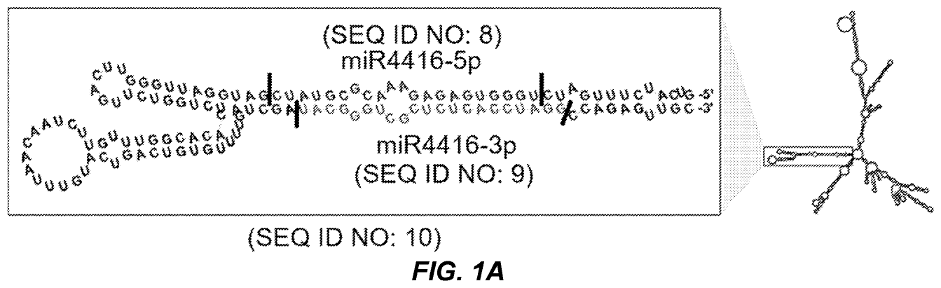

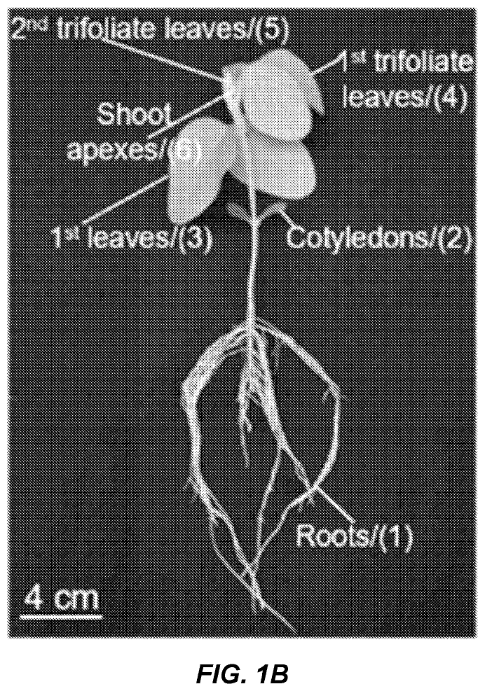

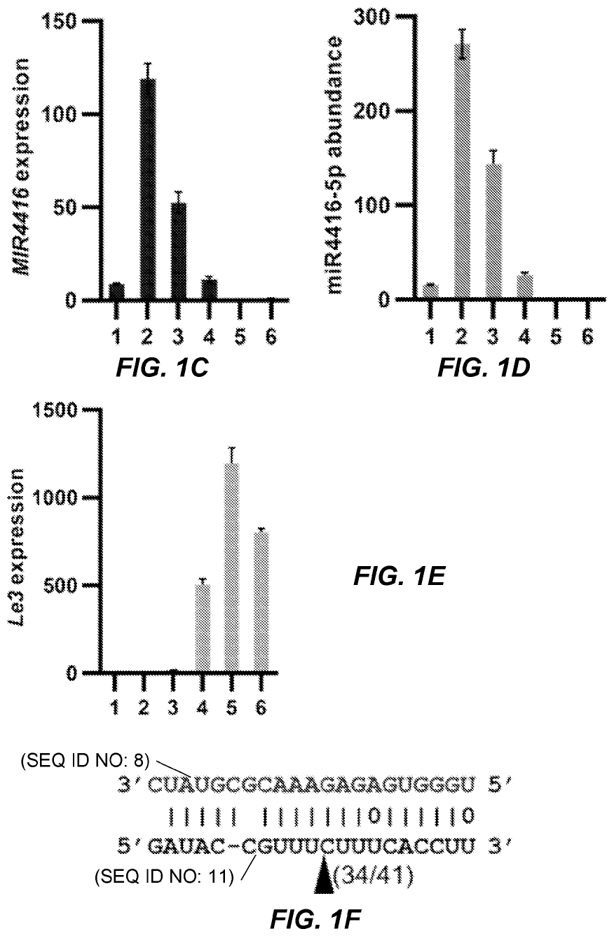

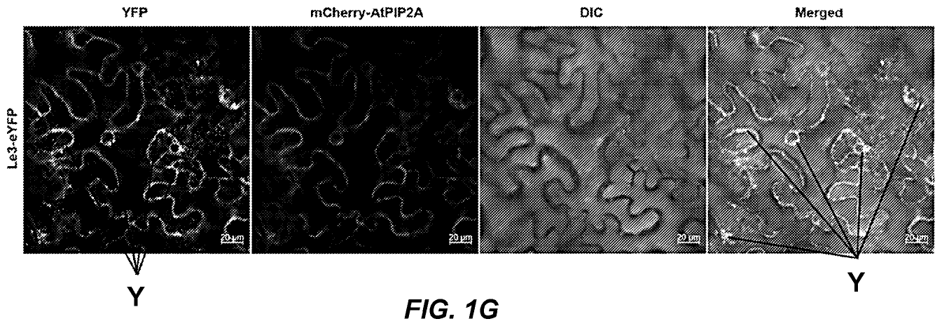

To identify the true miR4416b precursor, the miR4416b sequence was searched against the reference genome to identify a second, and the only other, perfect match in the entire genome, which is flanked by a site that perfectly matches miR4416 on chromosome 19 (FIG. 1A). It was determined that miR4416b and miR4416 are actually the 5p- and 3p-miRNAs produced by the same miRNA precursor MIR4416 located at this chromosomal locus and are thus re-designated miR4416-5p and miR4416-3p, respectively, according to widely-adopted miRNA nomenclature, to avoid confusion. Meyers et al., Criteria for annotation of plant MicroRNAs, Plant Cell 20:3186-3190 (2008).

To test miR4416-5p and Le3 levels in roots and nodules, Williams 82 seeds were sterilized with chlorine gas overnight and then germinated in sterilized vermiculite and sand mixture (3:1) for 7 days before either inoculation with a rhizobium culture or treatment with the MAG medium without the rhizobia. Di et al., Production of transgenic soybean lines expressing the bean pod mottle virus coat protein precursor gene, Plant Cell Reports 15:746-750 (1996). For testing responses to rhizobium, root hair cells were assessed in both the uninoculated and inoculated roots 6, 12, 24, 48, and 72 hours post inoculation/MAG medium treatment using quantitative reverse transcription-polymerase chain reaction (qRT-PCR) and stem-loop RT-qRT-PCR following a protocol described in Libault et al., Complete transcriptome of the soybean root hair cell, a single-cell model, and its alteration in response to Bradyrhizobium japonicum infection, Plant Physiology 152:541-552 (2010). Additionally, relative abundance of miR4416-5p/-3p was evaluated. Varkonyi-Gasic et al., Protocol: a highly sensitive RT-PCR method for detection and quantification of microRNAs, Plant Methods 3:12 (2007). The specificity of stem-loop RT-qRT-PCR was confirmed by sequencing the amplified fragments. The soybean gene GmELFIb was used as an internal reference to quantify the relative abundance of miR4416-5p determined by stem-loop RT-qRT-PCR from three biological replicates.

In the V1-stage soybean plants that were not inoculated with rhizobia (FIG. 1B), MIR4416 was expressed at the highest levels in cotyledons and then first leaves (FIG. 1C), which is where the highest abundance of miR4416-5p was also detected (FIG. 1D). In contrast, the putative miR4416-5p target, Glyma.02G156800, was expressed at relatively low levels in these two tissues (FIG. 1E). As expected, the mRNAs of Glyma.02G156800 were cleaved at the predicted miR4416-5p target sites (FIG. 1F), supporting that miR4416-5p represses the expression of Glyma.02G156800 through post-transcriptional regulation.

Glyma.02G156800, previously named Le3, encodes soybean lectin protein with an unknown function. Chragh et al., Le4 is an epicotyl preferential homologue of the soybean seed-specific Le1 lectin and the vegetative Le3 lectin genes, Plant Molecular Biology Reporter 33:1779-1789 (2015).

To examine the subcellular localization of Le3, full-length CDS was PCR amplified using gene-specific primers (Table 4, FIG. 13) and cloned into binary vector pCNHP-eYFP, which expressed fusion protein with C-terminal enhanced yellow fluorescent protein (eYFP). After sequencing verification, the vector was transformed into A. tumefaciens strain EHA105. A single colony was picked and cultured at 28° C. in 3 mL of Luria-Bertani (LB) medium supplied with 50 mg/L rifampicin and 50 mg/L kanamycin to OD₆₀₀ of about 2.0. The bacterial culture was pelleted, washed with 10 mM MgCl₂ and a MES (2-(N-morpholino) ethanesulfonic acid) (pH 5.7) solution containing 200 μM acetosyringone, and incubated in the same solution for additional 2 hours at room temperature.

Bacteria containing mCherry-labeled plasma membrane marker-encoding vector (ABRC stock: CD3-1008) was co-infiltrated with bacteria containing Le3-encoding vector. Before filtration, bacteria cultures were mixed to reach a final OD₆₀₀ of 0.6 for each of the cultures used. The suspension was injected into the abaxial surface of 4-6-week-old Nicotiana benthamiana leaves with a needleless syringe. 72 hours after infiltration, the fluorescent signals in detached leaves were imaged using a Zeiss LSM-880 laser-scanning confocal microscope (Zeiss, Thornwood, NY). The excitation wavelength and emission bandwidth were recorded for each fluorescent protein were optimized by the default presets in the ZEN 2.6 software (Zeiss) and were as follows: eYFP (excitation 514 nm, emission 519-583 nm), mCherry (excitation 561 nm, emission 580-651 nm).

It was determined that Le3 protein was localized in the plasma membrane (FIG. 1G). Unlike the soybean seed-specific lectin (SBL) gene Le1 (Glyma.02G012600), which is exclusively expressed in developing seeds and expressed at the highest level in the early maturation stage, Le3 was predominantly expressed in young leaves, flowers, and developing pod shells and is thus referred to as a vegetative lectin (SVL) gene. Chragh et al. (2015), supra. In addition to these shoot issues, a low level of expression of Le3 was detected in the roots, where about 83% of its mRNAs were cleaved by miR4416-5p (FIG. 1F). This supports miR4116-5p represses soy bean nodulation through post-transcriptional regulation of Le3 in root hairs.

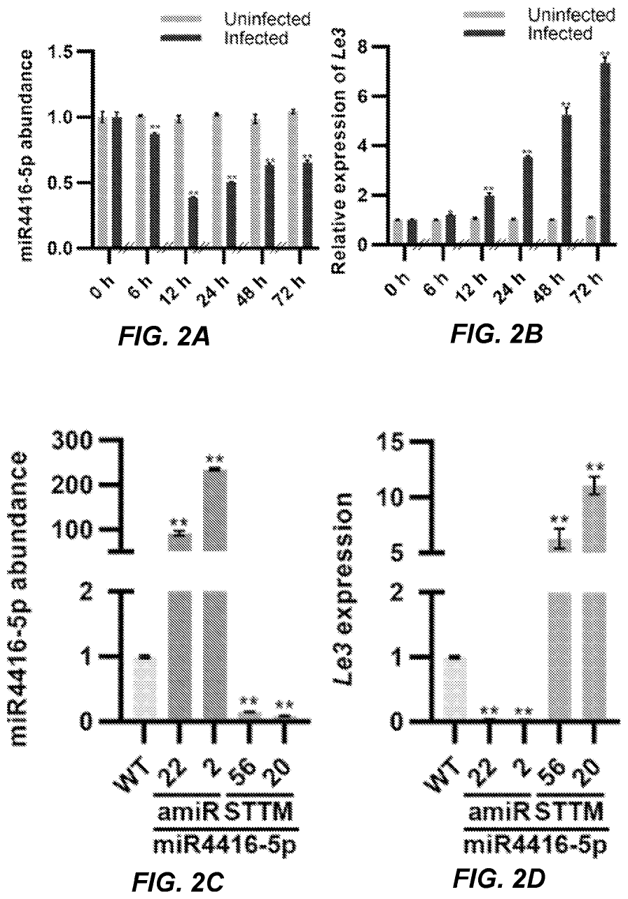

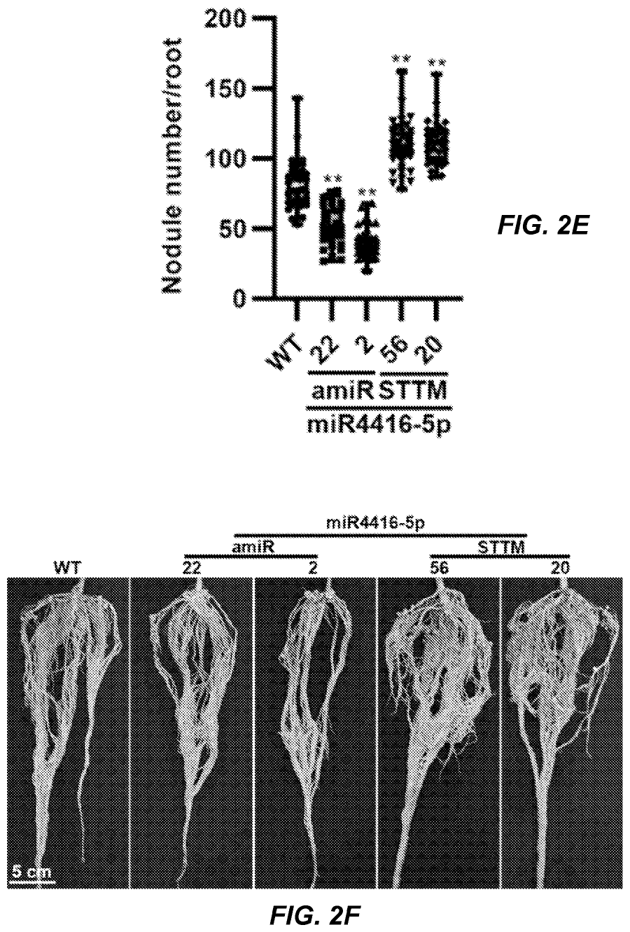

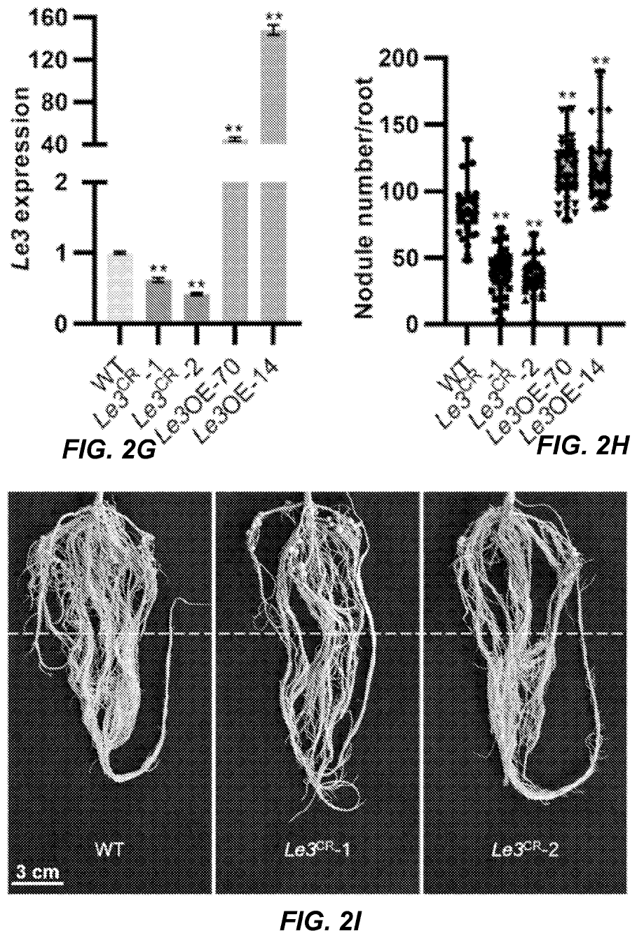

Further, it was determined that the abundance of miR4416-5p in the Williams 82 root hairs decreased upon the inoculation with B. diazoefficiens strain USDA110 (FIG. 2A). Correspondingly, the expression level of its target gene Le3 was increased upon the inoculation (FIG. 2B), suggesting that miR4416-5p is likely to be involved in the systemic regulation of the initial stage of the nodulation-rhizobial infection-through regulating the expression of Le3.

### Example 2

### Functional Roles of miR4416-5p and its Target Gene

To investigate the functional role of miR4416-5p and its target gene, an artificial miRNA precursor aMIR4416-5p was constructed by replacing the miR172-5p and miR172-3p sequences from the soybean miR172 (i.e., miR172-5p) precursor MIR172 with miR4416-5p and its complementary sequence, respectively (FIG. 6A). Briefly, a pEGAD vector was used to develop an artificial microRNA vector following a protocol described previously described in Ren et al. (2019), supra. See, e.g., Niu et al., Expression of artificial microRNAs in transgenic Arabidopsis thaliana confers virus resistance, Nature Biotechnology 24:1420-1428 (2006). The applicable primer pairs as listed in Table 4 (FIG. 13) were held at 95° C. for 4 minutes and then cooled down to 16° C. to form dimers, which were then inserted into the Age I and BamH I-digested pEGAD vector, separately.

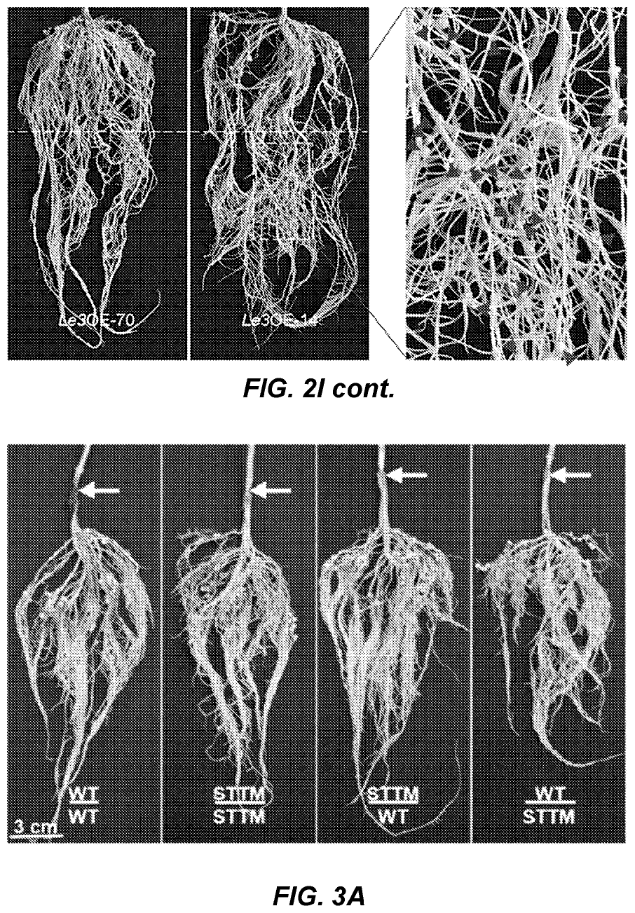

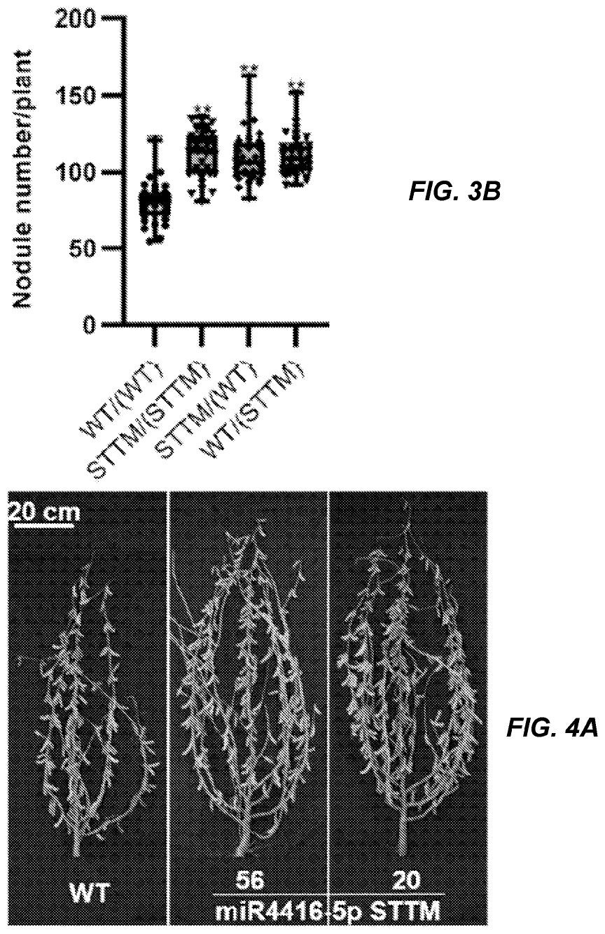

Further, transgenic short tandem target mimic (STTM) soybean roots were generated that silenced miR4416-5p (FIGS. 2C-3E). Briefly, the pEGAD vector was used to develop STTM following a protocol described previously described in Ren et al. (2019), supra. See also Yan et al. (2012), supra. Individual STTM modules were designed based on the miR4416-5p and each generated STTM vector silenced the corresponding miRNA in transgenic plants. The applicable primer pairs as listed in Table 4 (FIG. 13) were held at 95° C. for 4 minutes and then cooled down to 16° C. to form dimers, which were then inserted into the Age I and BamH I-digested pEGAD vector, separately. After confirming the correctness through Sanger sequencing, the pEGAD and miR4416-5p^{STTM} vectors were transformed into A. rhizogenes strain K599, separately. The hairy root transformation was generated via the A. rhizogenes-mediated protocol described in Ren et al. (2019), supra. Williams 82 was used as the transformation recipient.

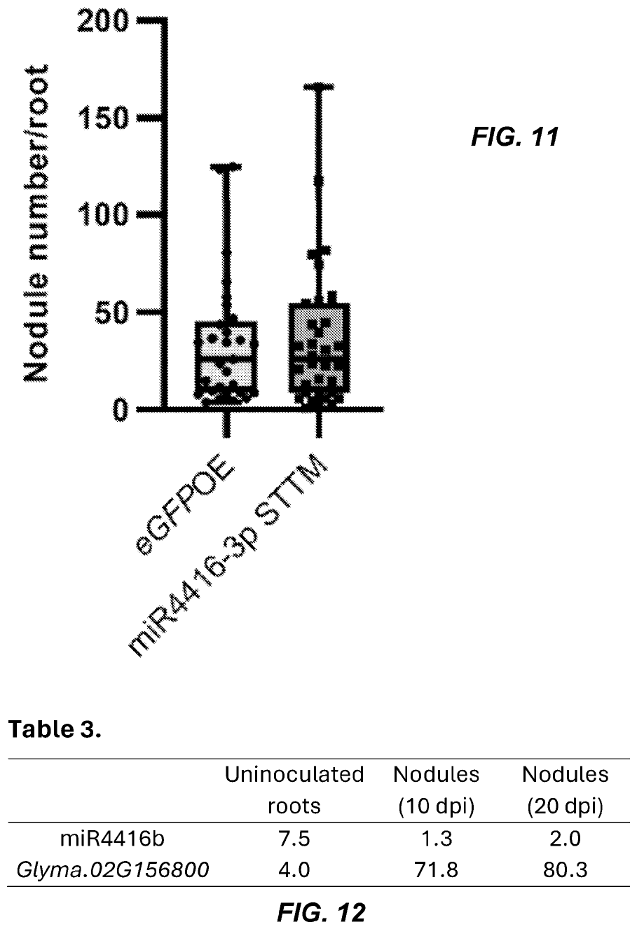

To test miR4416-5p and/or Le3 levels in nodules transformed with miR4416-3p ST™, the same setup was used as for Williams 82 nodules described above and the samples were harvested at 28 dpi for these stable transgenic plants or the wild-type control. No difference was observed in the nodule number of the STTM transgenic roots and eGFP transgenic roots (FIG. 11). These results support that it is miR4416-5p, rather than miR4416-3p, which regulates nodulation.

After confirming the correctness of the construct through Sanger sequencing, the vector was transformed into Agrobacterium tumefaciens strain EHA105. The stable transgenic soybean plants were then generated via A. tumefaciens-mediated cotyledonary node protocol/whole plant transformation to produce artificial miR4416-5p (amiR4416-5p) under the control of the cauliflower mosaic virus (CaMV) 35S promoter. Song et al., Screening Chinese soybean genotypes for Agrobacterium-mediated genetic transformation suitability, J Zhejiang University, Science B 14:289-298 (2013). Williams 82 was used as the transformation recipient and set as wild-type for phenotyping. Transgenic plants were identified by PCR of unique sequences from the vectors and further validated by the sequencing of the PCR fragments. Both the amiR4416-5p transgenic lines and non-transgenic control were then inoculated with B. diazoefficiens strain USDA110.

Root hair cells were assessed in both the transgenic and non-transgenic groups 6, 12, 24, 48, and 72 hours post inoculation using qRT-PCR and stem-loop RT-qRT-PCR, following a protocol described in Libault et al. (2010), supra. To test miR4416-5p and/or Le3 levels in nodules transformed with miR4416-5p artificial microRNA or the wild-type control, the same setup was used as for Williams 82 nodules described above and the samples were harvested and assessed at 28 dpi for these stable transgenic plants or the wild-type control.

In the transgenic lines, abundant miR4416-5p was produced (FIG. 2C), the expression level of Le3 was reduced (FIG. 2D), and fewer nodules were produced as compared to the non-transgenic control (FIGS. 2E and 2F), supporting that miR4416-5p is a negative regulator of nodulation.

### Example 3

### Interaction Between Le3 and miR4416-5p

To assess if Le3 acts as a positive regulator of nodulation, for example, via the cleavage of Le3 mRNAs by mi4416-5p and to align with the observed negative association between the abundance of miR4416-5p and the expression level of Le3, Williams 82 and CRISPR/Cas9 was used to generate Le3 mutants, which were then inoculated with B. diazoefficiens strain USDA110.

Primarily, the cleavage site of Le3 from the miR4416-5p was identified by 5′ RNA ligase-mediated rapid amplification of cDNA ends (RLM-RACE) using the GeneRacer™ Kit (Invitrogen Corporation, Waltham, MA) following the manufacture's protocol and as generally described above. Briefly, GeneRacer™ RNA Oligo was ligated to the 5′ phosphate of truncated mRNA and non-mRNA, to eliminate intact full-length mRNA from subsequent reverse transcription. First-strand cDNA was obtained by reverse transcription of the ligated RNA using the GeneRacer™ Oligo dT Primer. The cDNA samples were amplified by nested PCR, then the PCR products were cloned into the pCR™4Blunt-TOPO™ vector (Invitrogen Corporation, Waltham, MA) and sequenced at Eurofins Genomics (Louisville, KY) to determine the distribution and frequencies of cleaved fragments that define miR4416-5p guided cleavage sites within the predicted target Le3. This provided insight into the mechanism of action of the miRNA mi4416-5p.

Next, Le3 mutants were generated using known protocols, which included two Le3-genome edited mutants that included premature stop mutations (Le3^{CR-1} and Le3^{CR-2}) (FIG. 8) and two Le3-overexpressing mutants (Le3OE-70 and Le3OE-14) (which included a CaMV 35S promoter).

For generation of the premature stop mutation mutants Le3^{CR-1} and Le3^{CR-2}, the pGES201 vector was used to develop gRNA-Cas9 expression vectors following a protocol described previously. Bai et al., Generation of a multiplex mutagenesis population via polled CRISPR-Cas9 in soya bean, Plant Biotechnology J 18:721-731 (2020). sgRNA for editing the gene Le3 was designed using CRISPRdirect, a web-based guided RNA design software. The applicable primer pair listed in Table 4 (FIG. 13) was used, and the insert was inserted into the Bsa I-digested pGES201 vector. Naito et al., CRISPRdirect: software for designing CRISPR/Cas guide RNA with reduced off-target sites, Bioinformatics 31:1120-1123 (2015). This construct was subsequently used for transformation to obtain the Le3^{CR-1} and Le3^{CR-2} mutants.

For generation of the Le3 overexpressing mutants Le3OE-70 and Le3OE-14, the Le3 coding sequence (CDS) was amplified by the applicable primers listed in Table 4 (FIG. 13), and then integrated into the pRTL2 digested by Nco I and Xba I to develop the overexpression vector. The pRTL2 contained the cauliflower mosaic virus (CaMV) 35S promoter, the tobacco etch virus translational enhancer upstream of the Nco I site and the 35S termination sequence downstream of the Xba I site to facilitate expression in plant cells. Carrington & Freed, Cap-independent enhancement of translation by a plant potyvirus 5′ nontranslated region, J Virology 64:1590-1597 (1990). This construct was subsequently used for transformation to obtain the Le3OE-70 and Le3OE-14 mutants.

Briefly, after confirming the correctness through Sanger sequencing of each of the mutant constructs, the above-mentioned vectors were transformed into A. tumefaciens strain EHA105. The stable transgenic soybean plants were generated via A. tumefaciens-mediated cotyledonary node protocol as described in Song et al (2013), supra. Williams 82 was used as the transformation recipient and set as wild-type for phenotyping. Transgenic plants were identified by PCR of unique sequences from respective vectors and further validated by sequencing the PCR fragments.

To test miR4416-5p and/or Le3 levels in nodules transformed with Le3-genome editing (Le3^{CR}), Le3-overexpressing (Le3OE), or the wild-type control, the same setup was used as for the Williams 82 nodules described above, and the samples were harvested at 28 dpi for these stable transgenic plants or the wild-type control. Namely, Williams 82 seeds were sterilized with chlorine gas overnight and then germinated in sterilized vermiculite and sand mixture (3:1) for 7 days before inoculation with B. diazoefficiens strain USDA110. Expression levels of Le3 and miR4416-5p were then assessed.

In the Le3 mutants Le3^{CR-1} and Le3^{CR-2} with premature stop mutations, the expression levels of Le3 were lower as compared to that of the wild-type control (FIG. 2G) and the mutants produced fewer nodules (FIGS. 2H and 2I). In the Le3-overexpression lines Le3OE-70 and Le3OE-14, the expression levels of the transgenic Le3 were higher than that of the non-transgenic control (FIG. 2G), and the transgenic lines yielded more nodules (FIGS. 2H and 2I). Collectively, the results support that Le3 is a nodulation activator and is directly regulated by miR4416-5p through mRNA cleavage.

### Example 4

### miR4416-5p vs. miR4416-3p

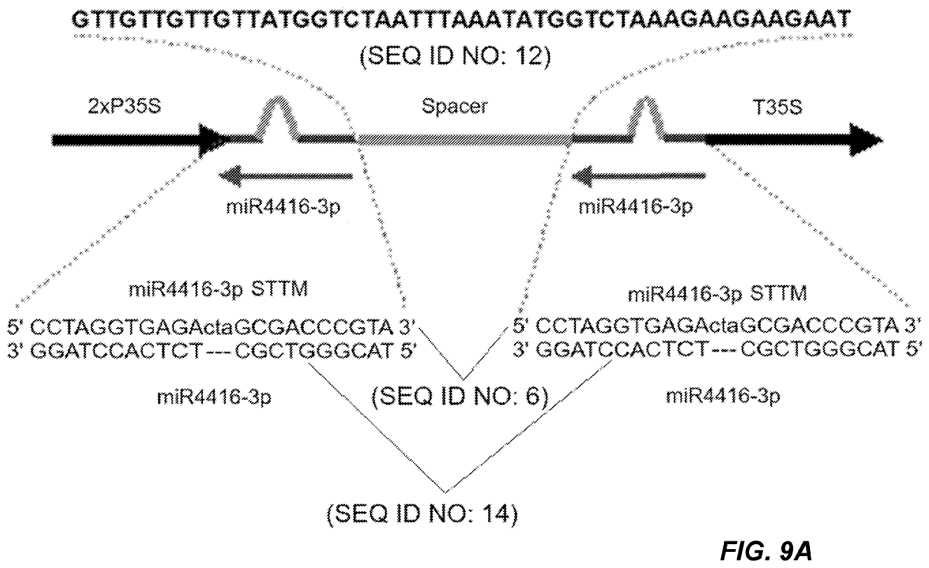

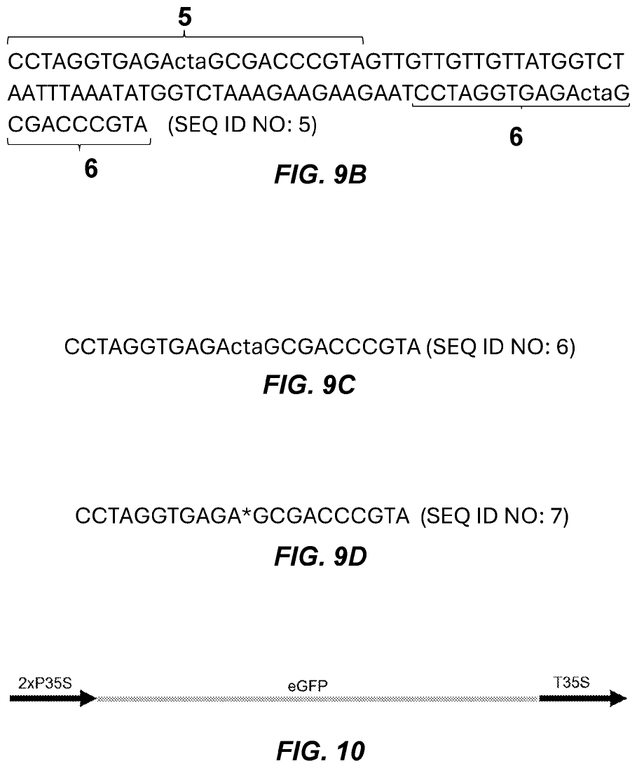

A previous study reported that miR4416 (i.e., miR4416-3p) in root hairs was responsive to B. diazoefficiens strain USDA110. Yan et al., Identification and functional characterization of soybean root hair microRNAs expressed in response to B. japonicum infection, Plant Biotechnology J 14:332-341 (2016). Additionally, it was demonstrated that ectopic expression of MIR4416 in Agrobacterium rhizogenes-induced transgenic hairy roots resulted in a reduction in nodule number. As MIR4416 was not annotated to be the precursor of miR4416-5p at that time, miR4416-3p was regarded as the negative regulator of nodulation. To address if miR4416-3p regulates nodulation (in addition to Le3), using Williams 82, transgenic short tandem target mimic (STTM) soybean roots were generated that silenced miR4416-3p (FIG. 9A), as well as transgenic roots that expressed the enhanced green florescence protein (eGFP) (FIG. 10). The two constructs differed only in the portions of STTM and eGFP.

Briefly, the pEGAD vector was used to develop STTM following a protocol described previously described in Ren et al. (2019), supra. See also Yan et al. (2012), supra. Individual STTM modules were designed based on the miR4416-5p/-3p and each generated STTM vector silences the corresponding miRNA in transgenic plants. The applicable primer pairs as listed in Table 4 (FIG. 13) were held at 95° C. for 4 minutes and then cooled down to 16° C. to form dimers, which were then inserted into the Age I and BamH I-digested pEGAD vector, separately.

After confirming the correctness through Sanger sequencing, the pEGAD and miR4416-3p^{STTM} vectors were transformed into A. rhizogenes strain K599, separately. The hairy root transformation was generated via the A. rhizogenes-mediated protocol described in Ren et al. (2019), supra. Williams 82 was used as the transformation recipient. Transgenic roots were identified by PCR of unique sequences from pEGAD and miR4416-3p^{STTM} and further validated by sequencing the PCR fragments.

To test miR4416-5p and/or Le3 levels in nodules transformed with miR4416-3p^{STTM}, the same setup was used as for Williams 82 nodules described above and the samples were harvested at 28 dpi for these stable transgenic plants or the wild-type control. No difference was observed in the nodule number of the STTM transgenic roots and eGFP transgenic roots (FIG. 11). These results support that it is miR4416-5p, rather than miR4416-3p, which regulates nodulation.

### Example 5

### miR4416-5p in the Roots Transported from the Shoots to Regulate Nodulation

The expression levels of MIR4416 in leaves, particularly cotyledons and developed leaves, are extremely high, while its level of expression in roots is low, suggesting that a small portion of miR4416-5p accumulated in roots may be produced locally. To assess the contribution of shoot-transported miR4416-5p to nodulation regulation, a grafting experiment was conducted using transgenic plants expressing the STTM lines described above, namely, where miR4416-5p was silenced and a wild-type control (Williams 82), both at the V0 developmental stage of soybean plants when their unfolded unifoliolate leaves emerged. Both were then inoculated with B. diazoefficiens strain USDA110.

The grafting studies consisted of four distinct scion/rootstock combinations-Williams 82/Williams 82 and miR4416-5p^{STTM}/miR4416-5p^{STTM} homografted plants, and miR4416-5p^{STTM}/Williams 82 and Williams 82/miR4416-5p^{STTM} heterografted plants. Grafting was performed as previously described in Pantalone et al., Soybean PI 416937 root system contributes to biomass accumulation in reciprocal grafts, Agronomy J 91:840-844 (1999), with minor modification.

Briefly, seeds of Williams 82 and miR4416-5p^{STTM} were sterilized with chlorine gas overnight and then germinated in sterilized vermiculite and sand mixture (3:1). Grafting was initiated 10 days after planting (V0 stage), when the apical meristem of seedlings had reached a height of 12 cm above the soil surface. The hypocotyl was transversely severed with a razor approximately 2 cm below the cotyledon. The lower portion remained in the soil to become the rootstock and the upper portion became the scion. A vertical incision was made with a razor to a depth of approximately 0.75 cm into the top center of the rootstock.

The scion was razor-trimmed to a V-wedge, which was inserted into the rootstock incision. The graft union was wrapped with Parafilm® M Laboratory Film (Amcor, Zurich, Switzerland). Grafts were placed under greenhouse benches, out of direct sunlight. One week later, successfully grafted plants were inoculated with the B. diazoefficiens USDA 110 and nodules were counted at 28 dpi. Four each of the four scion/rootstock combinations, three biological replicates were conducted, and in each replicate, 12 grafts for each combination were monitored.

The two heterografted plants and the miR4416-5p^{STTM}/miR4416-5p^{STTM} plants all produced more nodules than Williams 82/Williams 82, but no significant difference in nodule numbers was seen among the three combinations involving miR4416-5p^{STTM} (FIGS. 3A and 3B). These results support that it is the shoot-generated miR4416-5p that regulates nodulation systemically, while root-generated miR4416-5p has no or minimal effects on nodulation.

### Example 6

### Effects of miR4416-5p and Le3 on Plant Productivity, Yield Components, and Seed Compositions

The findings that miR4416-5p in the shoots is barely detected in developing tissues where Le3 is highly expressed, and that shoot-to-root transported miR4416-5p is negatively regulated by rhizobia in root hairs where Le3 is upregulated to promote nodulation, suggest that the miR4416-5p-Le3 module may execute dual roles in regulating plant growth and development without negative feedback with symbiotic nitrogen fixation (SNF). To test this, a field test of the miR4416-5p^{STTM} lines and Le3 overexpression lines were conducted for various yield components traits (plant height, primary branch number, main stem node number, pod number, seed number, and 100-seed weight per plant) as well as seed composition traits (protein and oil contents).

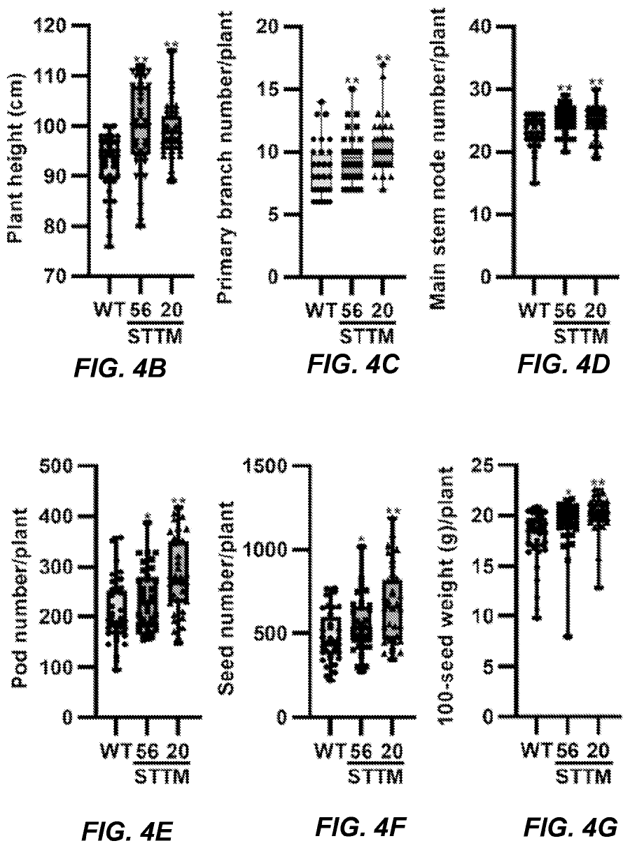

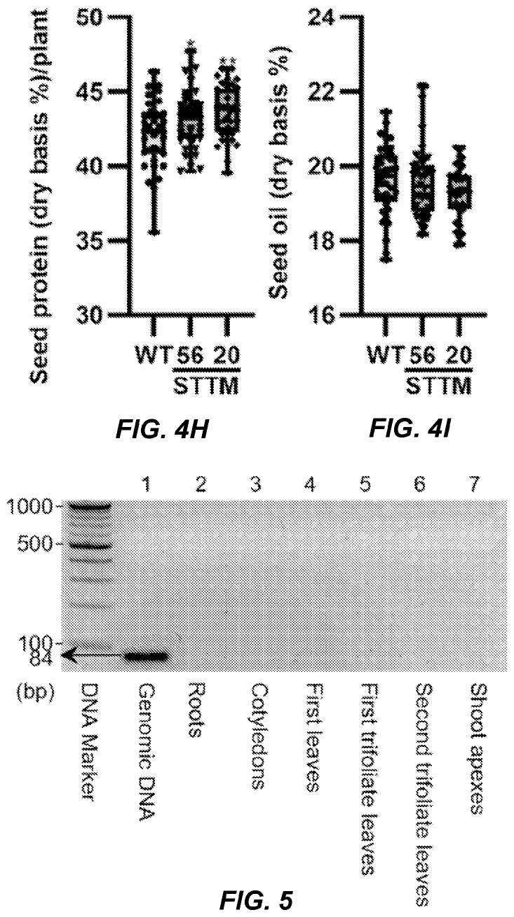

For the STTM lines, T2 (i.e., the second generation of a specific transgenic event) plants (FIG. 4A) and T3 seeds harvested from the T2 plants were measured. For the Le3 overexpression lines, T1 (i.e., the first generation of a specific transgenic event) plants and T2 seeds from the T1 plants were measured.

The STTM lines showed improvement for all yield component traits (FIGS. 4B-4G). For example, the seed number per plant was increased up to an average of 35.4% for a STTM line. Notably, the increase of seed number per plant didn't result in a decrease of 100-seed weight, which, instead, was increased up to an average of 8.4% for this STTM line, representing about a 47% increase in seed yield as compared to the wild-type Williams 82.

Seed protein and oil contents in the same STTM line were measured using Near-infrared (NIR) spectroscopy. The seed protein was increased by 3.7% without a statistically detectable reduction in seed oil content. The yield increase gained by individual plants reflects the yield increase for the STTM lines, since the plots of the STTM T2 plants from different T1 seeds were arranged randomly in the same field.

Given that only a small number of T1 seeds were obtained from the Le3-overexpression transgenic events in the greenhouse, there were not enough T1 plants available for meaningful measurement of the yield component traits in the field. Nevertheless, a larger increase in seed protein content and a decrease in seed oil content were observed in the Le3-overexpression T2 seeds. These similar patterns of changes in seed compositions as seen between the miR4416-5p^{STTM} lines and the Le3-overexpression lines grown in the field, along with the similar patterns of changes in nodulation and plant growth as observed between the earlier generation of these two-types of transgenic lines grown in the greenhouse, support that an increase in seed yield can be achieved or facilitated by overexpressing Le3.

### Example 7

### Yield and Seed Compositions in T3-Generation Stable STTM Lines

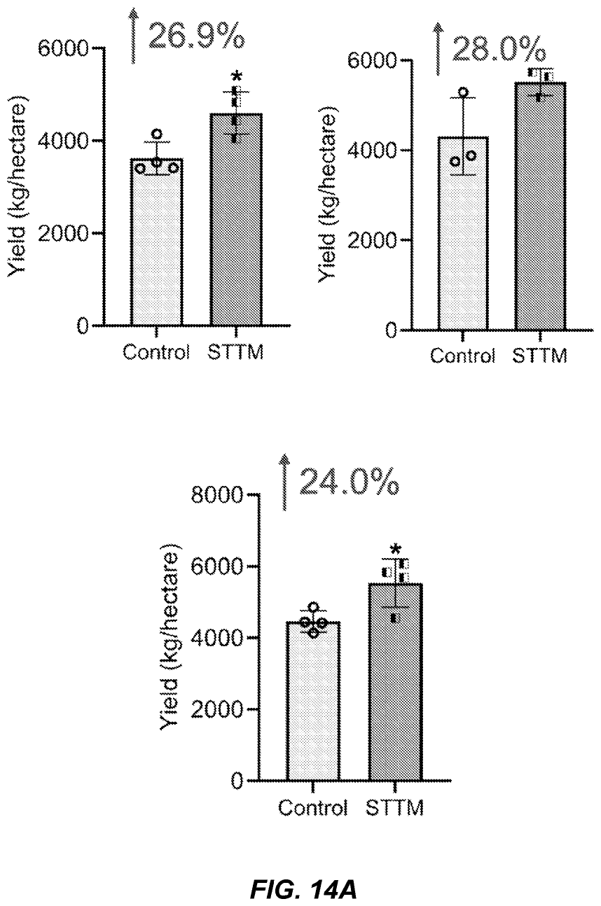

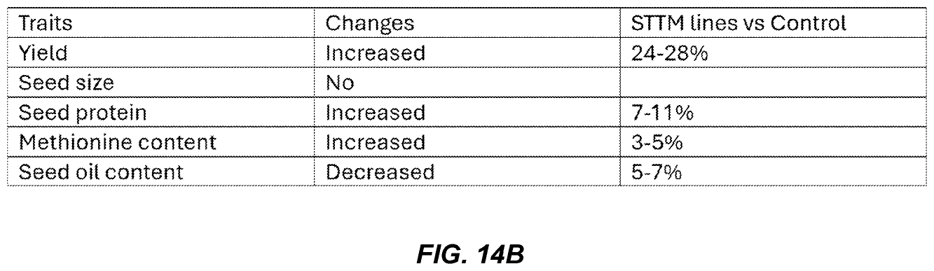

An additional field test of the miR4416-5p^{STTM} lines were conducted for various yield components traits (plant height, primary branch number, main stem node number, pod number, seed number, and 100-seed weight per plant) as well as seed composition traits (protein and oil contents), in this instance with T3 plants. The yield plot size in each replicate for the test was four rows, 30 inches apart, and 8 feet long, and three experimental replicates were conducted in Purdue Agronomy Farm, West Lafayette, Indiana in 2023. The middle two rows were harvested to measure yield. Yield was measured in three independent STTM lines in the Williams 82 genetic background and compared with wild-type Williams 82 (control) (FIG. 14A). Phenotypic changes were observed and measured, and seed protein and oil contents were measured using NIR spectroscopy (Perten DA 7250, PerkinElmer, US LLC, Sheltion, CT).

FIG. 14B shows the graphical data supporting that the ranges of yield increased, and the seed compositions changed, in the three STTM lines as compared with the control.

### Example 8

### Mutants Generated from Gene Editing

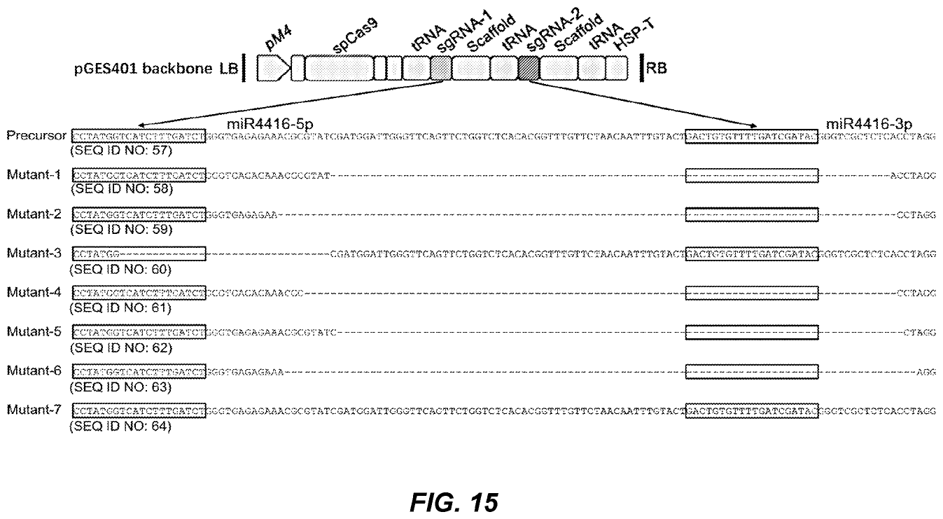

Additional mutants were generated using known CRISPR/Cas9 gene editing techniques. Generally, for each mutant, a construct pGES401 backbone and two gRNA sequences (nucleotides shown within the frames indicated in FIG. 15) was used to edit the miR4416-5p and miR4416-3p precursor gene MIR4416. CRISPR editing was employed to delete nucleic acids of the precursor (deletions represented by dashed lines in FIG. 15), although in each case at least a portion of the miR4416-5p sequence was preserved.

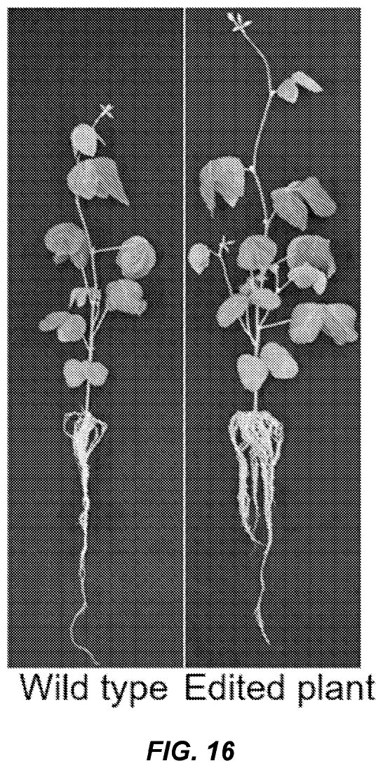

Briefly, a pGES401 vector was used to develop gRNA-Cas9 expression vectors to edit the miR4416 precursor gene following a protocol described previously. Bai et al. (2020), supra. This construct was subsequently used for transformation to obtain gene-editing plants which showed increased plant growth as compared with corresponding wild-type plants (FIG. 16).

1. A leguminous plant comprising at least one mutation, the at least one mutation comprising:
a first mutation that upregulates expression of a Le3 gene in the leguminous plant as compared to expression in a corresponding wild-type leguminous plant; and/or
a second mutation that downregulates or silences expression of microRNA (miRNA) miR4416-5p or miR4416-3p.

2. The leguminous plant of claim 1, wherein the at least one mutation comprises both the first mutation and the second mutation.

3. The leguminous plant of claim 1, wherein the plant is a soybean cultivar.

4. The leguminous plant of claim 1, wherein the upregulation is facilitated by the incorporation of one or more exogenous Le3 genes into a genome of the plant.

5. The leguminous plant of claim 4, wherein the one or more exogenous Le3 genes are integrated at a single genomic locus.

6. The leguminous plant of claim 4, wherein the one or more exogenous Le3 genes comprises at least two exogenous Le3 genes that are integrated at multiple genomic loci.

7. The leguminous plant of claim 1, wherein upregulation is facilitated by downregulating or silencing expression of miR4416-5p or miR4416-3p.

8. The leguminous plant of claim 1, wherein the Le3 gene is operably linked to a promoter that upregulates expression of the Le3 gene.

9. The leguminous plant of claim 1, wherein the upregulation is facilitated by the incorporation of one or more gene regulatory elements.

10. The leguminous plant of claim 1, wherein the plant is a plant cell.

11. The leguminous plant of any one of claims 1-10, wherein the second mutation is encoded by SEQ ID NO: 3 or SEQ ID NO: 4.

12. The leguminous plant of claim 1, wherein the second mutation is encoded by one or more modified miR4416-5p sequences or one or more modified miR4416-3p sequences, and, optionally:
the modified miR4416-5p sequences each independently comprise a miR4416-5p short tandem target mimic (STTM) sequence or a sequence of wild-type miR4416-5p comprising an insertion or deletion therein, and
the modified miR4416-3p sequences each comprise a miR4416-3p STTM sequence or a sequence of wild-type miR4416-3p comprising an insertion or deletion therein.

13. The leguminous plant of claim 12, wherein the modified miR4416-5p sequence comprises a wild-type miR4416-5p sequence with a bulge sequence inserted therein.

14. The leguminous plant of claim 12, wherein the modified miR4416-3p sequence comprises a wild-type miR4416-3p sequence with a bulge sequence inserted therein.

15. The leguminous plant of claim 13 or 14, wherein the bulge sequence is or comprises CTA.

16. The leguminous plant of claim 1, wherein the second mutation is encoded by at least two modified miR4416-5p sequences and/or at least two modified miR4416-3p sequences, each of the modified miR4416-5p or miR4416-3p sequences separated by a spacer, wherein, optionally:
each of the at least two modified miR4416-5p sequences independently comprises SEQ ID NO: 3 or SEQ ID NO: 4, or a functional variant of SEQ ID NO: 3 or SEQ ID NO: 4; and/or
each of the at least two modified miR4416-3p sequences independently comprises SEQ ID NO: 6 or SEQ ID NO: 7, or a functional variant of SEQ ID NO: 6 or SEQ ID NO: 7.

17. The leguminous plant of claim 16, wherein the spacer is of a length sufficient to allow for binding between two of the modified miR4416-5p sequences or two of the modified miR4416-3p sequences in a loop structure.

18. The leguminous plant of claim 16, wherein the spacer comprises between about 3 to about 99 nucleic acids and is optionally about 48 nucleic acids.

19. The leguminous plant of claim 1, wherein the at least one mutation results in interference with a miRNA-Le3 interaction in the plant.

20. The leguminous plant of any one of claims 1-10, 12-14, and 16-19, wherein the at least one mutation is encoded by SEQ ID NO: 1 or a functional variant of SEQ ID NO: 1.

21. An expression construct comprising:
a first nucleic acid sequence that upregulates expression of a Le3 gene in a leguminous plant as compared to wild-type expression in a corresponding wild-type leguminous plant; and/or
a second nucleic acid sequence that downregulates or silences expression of microRNA (miRNA) miR4416-5p or miR4416-3p.

22. The expression construct of claim 21, wherein the expression construct comprises both the first nucleic acid sequence and the second nucleic acid sequence.

23. The expression construct of claim 21, comprising SEQ ID NO: 1, 2, or 5.

24. The expression construct of claim 21, comprising the first nucleic acid sequence, wherein the first nucleic acid sequence encodes two or more Le3 genes.

25. The expression construct of claim 24, wherein the two or more Le3 genes are encoded at a single genomic locus.

26. The expression construct of claim 24, wherein the two or more Le3 genes are encoded at multiple genomic loci.

27. The expression construct of claim 21, comprising the first nucleic acid sequence, wherein the first nucleic acid sequence comprises one or more gene regulatory elements.

28. The expression construct of claim 27, wherein the one or more gene regulatory elements comprise at least a promoter for upregulating expression of the Le3 gene as compared to wild-type expression.

29. The expression construct of claim 21, comprising the second nucleic acid sequence, wherein the second nucleic acid sequence comprises SEQ ID NO: 3 or SEQ ID NO: 4.

30. The expression construct of claim 21, comprising the second nucleic acid sequence, wherein the second nucleic acid sequence comprises one or more modified miR4416-5p sequences or one or more modified miR4416-3p sequences, and, optionally wherein:
at least one of the modified miR4416-5p sequences comprises a miR4416-5p short tandem target mimic (STTM) sequence or a sequence of wild-type miR4416-5p comprising an insertion or deletion therein; and
at least one of the modified miR4416-3p sequences comprises a miR4416-3p STTM sequence or a sequence of wild-type miR4416-3p comprising an insertion or deletion therein.

31. The expression construct of claim 30, wherein the one or more modified miR4416-5p sequences comprises a wild-type miR4416-5p sequence with a bulge sequence inserted therein.

32. The expression construct of claim 30, wherein the one or more modified miR4416-3p sequences comprises a wild-type miR4416-3p sequence with a bulge sequence inserted therein.

33. The expression construct of claim 31 or 32, wherein the bulge sequence is or comprises CTA.

34. The expression construct of claim 21, comprising the second nucleic acid sequence, wherein the second nucleic acid sequence encodes two or more miR4416-5p or two or more modified miR4416-3p, wherein each of the modified miR4416-5p or miR4416-3p sequences is separated by a spacer.

35. The expression construct of claim 34, wherein the spacer is of a length sufficient to allow for binding between two of the modified miR4416-5p sequences or two of the modified miR4416-3p sequences in a loop structure.

36. The expression construct of claim 34, wherein the spacer comprises between about 3 to about 99 nucleic acids and is optionally about 48 nucleic acids.

37. A method for enhancing leguminous plant growth comprising:
upregulating expression of a Le3 gene in a plant as compared to expression in a corresponding wild-type leguminous plant; and/or
downregulating or silencing expression of microRNA (miRNA) miR4416-5p, miR4416-3p, or both miR4416-5p and miR4416-3p in the plant as compared to expression in a corresponding wild-type leguminous plant.

38. The method of claim 37, wherein the method comprises both upregulating expression of a Le3 gene and downregulating or silencing expression of miR4416-5p or miR4416-3p.

39. The method of claim 37, wherein upregulating expression of a Le3 gene and/or downregulating or silencing expression of miR4416-5p and/or miR4416-3p comprises introducing one or more expression constructs of any one of claims 21-36 into the plant or incorporating modifications into a sequence of miR4416-5p and/or the sequence of miR4416-3p of the plant.

40. The method of claim 39, wherein upregulating expression of a Le3 gene comprises incorporating one or more exogenous Le3 genes into a genome of the plant.

41. The method of claim 40, wherein the one or more exogenous Le3 genes are integrated at a single genomic locus.

42. The method of claim 40, wherein the one or more exogenous Le3 genes comprises at least two exogenous Le3 genes that are integrated at multiple genomic loci.

43. The method of claim 37, wherein the Le3 gene is operably linked to a promoter that upregulates expression of the Le3 gene.

44. The method of claim 37, wherein upregulating comprises incorporating one or more gene regulatory elements into a genome of the plant.

45. The method of claim 37, wherein the plant is a plant cell.

46. The method of claim 37, wherein the downregulating or silencing expression of miR4416-5p and/or miR4416-3p comprises transforming the plant with a construct comprising SEQ ID NO: 3, SEQ ID NO: 4, SEQ ID NO: 6, SEQ ID NO: 7, or two or more of SEQ ID NOS: 3, 4, 6, or 7.

47. The method of claim 37, wherein downregulating or silencing expression of miR4416-5p and/or miR4416-3p comprises transforming the plant with an expression construct comprising one or more modified miR4416-5p sequences or one or more modified miR4416-3p sequences, and, optionally wherein:
at least one of the modified miR4416-5p sequences comprises a miR4416-5p short tandem target mimic (STTM) sequence or a sequence of wild-type miR4416-5p comprising an insertion or deletion therein; and
at least one of the modified miR4416-3p sequences comprises a miR4416-3p STTM sequence or a sequence of wild-type miR4416-3p comprising an insertion or deletion therein.

48. The method of claim 47, wherein the one or more modified miR4416-5p sequence comprises a wild-type miR4416-5p sequence with a bulge sequence inserted therein.

49. The method of claim 47, wherein the one or more modified miR4416-3p sequence comprises a wild-type miR4416-3p sequence with a bulge sequence inserted therein.

50. The method of claim 48 or 49, wherein the bulge sequence is or comprises CTA.

51. The method of claim 39, wherein introducing or incorporating is performed by gene editing techniques such as, optionally, CRISPR-Cas9, TALENs, or homologous recombination.

52. The method of claim 51, wherein incorporating comprises incorporating an insertion into a sequence of miR4416-5p or miR4416-3p of the plant.

53. The method of claim 39, wherein introducing one or more expression constructs into the plant further comprises transforming the leguminous plant with one or more of the expression constructs.

54. The method of claim 37, wherein plant yield is enhanced by at or more than about 20% as compared to plant yield of a corresponding wild-type plant.

55. The method of claim 37, wherein seed protein concentration is increased as compared to seed protein concentration in a corresponding wild-type plant.

56. The method of claim 39, wherein introducing or incorporating is performed prior to planting the plant or after planting the plant.

57. The method of any one of claims 37-56, further comprising inoculating, or having inoculated, the plant with at least one rhizobium to initiate nodulation.
